(19) United States
(12) Plant Patent Application Publication
(10) Pub. No.: US 2023/0037287 P1
(43) Pub. Date: Feb. 2, 2023

(54) PONGAMIA VARIETIES

(50) ;

(71) Applicant: Terviva, Inc., Oakland, CA (US)

(72) Inventors: Naidu Bodapati, Riverhills (AU); Marshall Mackay, Tewantin (AU); Naveen Sikka, Oakland, CA (US); David Harry, Philomath, OR (US)

(73) Assignee: Terviva, Inc., Oakland, CA (US)

(21) Appl. No.: 17/967,572

(22) Filed: Oct. 17, 2022

Related U.S. Application Data
(63) Continuation of application No. 17/409,256, filed on Aug. 23, 2021.
Application No. 17/409,256 is a continuation of application No. 17/061,410, filed on Oct. 1, 2020, now abandoned.
Application No. 17/061,410 is a continuation of application No. 16/602,144, filed on Aug. 14, 2019, now abandoned.
(60) Provisional application No. 62/722,102, filed on Aug. 23, 2018.

(51) Int. Cl. A01H 6/54

(52) U.S. Cl. PLT/216

(57) ABSTRACT

New and distinct varieties of Pongamia pinnata (L) Pierre trees (pongamia trees) are described herein.

### CROSS-REFERENCE TO RELATED APPLICATIONS

This application is a continuation of U.S. application Ser. No. 17/409,256, filed Aug. 23, 2021, which is a continuation of U.S. application Ser. No. 17/061,410, filed Oct. 1, 2020, which is a continuation of U.S. application Ser. No. 16/602,144, filed Aug. 14, 2019, which claims the benefit of U.S. Provisional Application No. 62/722,102, filed Aug. 23, 2018, each of which is incorporated herein by reference in its entirety.

Latin name: Botanical classification: The pongamia varieties described herein are botanically identified as Pongamia pinnata (L) Pierre.

Varietal denomination: The varietal denominations of the claimed varieties of pongamia as described herein are ‘K002’, ‘K006’, ‘K008’, ‘K009’, ‘K010’, ‘K011’, ‘K013’, ‘K022’, ‘K025’, ‘K141’, ‘K187’, ‘K202’, ‘K205’, ‘K206’, ‘K207’, ‘K208’, ‘K209’, ‘K211’, ‘K219’, ‘K268’, ‘K303’, ‘K309’, ‘K605’, ‘K607’, ‘K608’, ‘K609’, and ‘K610’.

### BACKGROUND OF THE INVENTION

The present disclosure relates to new and distinct varieties of pongamia trees. Pongamia is a species of tree that belongs to the Fabaceae family of plants. The botanical classification of pongamia is Pongamia pinnata (L) Pierre, although these plants are also known by a number of synonymous botanical classifications including, for example, Millettia pinnata (L) Panigrahi, Derris indica (Lam.) Bennet, Pongamia glabra Vent, and Cytisus pinnatus (L).

As a member of the Fabaceae family of plants, pongamia trees are legumes and are capable of fixing their own nitrogen. Pongamia can grow well in a variety of environmental conditions, including areas with malnourished soil. Pongamia seeds also contain a profile of lipids having a number of uses including, for example, commercial preparation into skin ointment and as a fuel source.

Pongamia trees are capable of growing in otherwise challenging environmental conditions and produce seed oils with beneficial properties. There exists a need for pongamia varieties that are stable, high yielding, and agronomically sound.

### SUMMARY OF THE INVENTION

The present disclosure relates to new and distinct pongamia (Pongamia pinnata (L) Pierre) varieties designated as ‘K002’, ‘K006’, ‘K008’, ‘K009’, ‘K010’, ‘K011’, ‘K013’, ‘K022’, ‘K025’, ‘K141’, ‘K187’, ‘K202’, ‘K205’, ‘K206’, ‘K207’, ‘K208’, ‘K209’, ‘K211’, ‘K219’, ‘K268’, ‘K303’, ‘K309’, ‘K605’, ‘K607’, ‘K608’, ‘K609’, and ‘K610’. Each of these pongamia varieties was discovered growing in a cultivated area in Queensland, Australia, wherein it and neighboring trees are grown for shade and other landscape amenities. The parentage of each of these pongamia trees is unknown, as is typical for trees cultivated in landscape plantings of this type.

The original ortet (donor tree) of each of these pongamia varieties was initially identified and selected primarily on the basis of its consistent production of abundant seed pods coupled with the high oil content of its seeds. Following initial selection of each variety, branch cuttings were collected from the donor tree and rooted by treating the cuttings with the rooting hormone indolebutyric acid (IBA) using either an aqueous solution of IBA (500 ppm) or a commercially available rooting powder. Dehydration of the cuttings was avoided by placing them in moistened rooting medium within a humid enclosed chamber. Rooting success varied from one collection event to another, which is normal for cuttings from mature trees. Typically 10% or more of the cuttings initiated root development within 3-4 weeks. Each of these pongamia varieties have been asexually reproduced in Queensland, Australia through rooted cuttings and remain stable and true-to-type following successive asexual propagations.

### DESCRIPTION OF THE DRAWINGS

The pongamia trees as described herein are illustrated by the accompanying photographs which show e.g. tree form, branches, foliage, leaves, trunk, pods, and seeds as specifically described below. The colors shown are as true as can reasonably be obtained by conventional photographic procedures.

FIG. 1A-FIG. 1G illustrate the plant or parts thereof of variety ‘K002’. FIG. 1A and FIG. 1B illustrate a seed pod and seeds of variety ‘K002’. FIG. 1C and FIG. 1D illustrate views of the whole tree of variety ‘K002’. FIG. 1E illustrates the tree trunk of variety ‘K002’. FIG. 1F illustrates the canopy of variety ‘K002’. FIG. 1G illustrates seed pods containing seeds of variety ‘K002’.

FIG. 2A-FIG. 2D illustrate the plant or parts thereof of variety ‘K006’. FIG. 2A and FIG. 2B illustrate views of the whole tree of variety ‘K006’. FIG. 2C illustrates the tree trunk of variety ‘K006’. FIG. 2D illustrates seed pods and seeds of variety ‘K006’.

FIG. 3A-FIG. 3C illustrate the plant or parts thereof of variety ‘K008’. FIG. 3A and FIG. 3B illustrate views of the whole tree of variety ‘K008’. FIG. 3C illustrates a branch with leaves and seed pods of variety ‘K008’.

FIG. 4A-FIG. 4F illustrate the plant or parts thereof of variety ‘K009’. FIG. 4A illustrates the whole tree of variety ‘K009’. FIG. 4B illustrates seed pods of variety ‘K009’. FIG. 4C illustrates the canopy of variety ‘K009’. FIG. 4D and FIG. 4E illustrate views of branches and seed pods of variety ‘K009’. FIG. 4F illustrates the tree trunk of variety ‘K009’.

FIG. 5A-FIG. 5D illustrate the plant or parts thereof of variety ‘K010’. FIG. 5A and FIG. 5B illustrate views of the whole tree of variety ‘K010’. FIG. 5C illustrates leaves of variety ‘K010’. FIG. 5D illustrates the tree trunk of variety ‘K010’.

FIG. 6A-FIG. 6B illustrate the plant or parts thereof of variety ‘K011’. FIG. 6A illustrates the whole tree of variety ‘K011’. FIG. 6B illustrates the tree trunk of variety ‘K011’.

FIG. 7A-FIG. 7C illustrate the plant or parts thereof of variety ‘K013’. FIG. 7A and FIG. 7B illustrate views of the whole tree of variety ‘K013’. FIG. 7C illustrates the tree trunk of variety ‘K013’.

FIG. 8A-FIG. 8B illustrate the plant or parts thereof of variety ‘K022’. FIG. 8A illustrates the whole tree of variety ‘K022’. FIG. 8B illustrates the tree trunk of variety ‘K022’.

FIG. 9A-FIG. 9E illustrate the plant or parts thereof of variety ‘K025’. FIG. 9A and FIG. 9B illustrate views of the whole tree of variety ‘K025’. FIG. 9C and FIG. 9D illustrate views of the canopy of variety ‘K025’. FIG. 9E illustrates the tree trunk of variety ‘K025’.

FIG. 10 illustrates the whole tree of variety ‘K141’.

FIG. 11A-FIG. 11J illustrate the plant or parts thereof of variety ‘K187’. FIG. 11A and FIG. 11B illustrate views of the whole tree of variety ‘K187’. FIG. 11C, FIG. 11D, FIG. 11E, and FIG. 11F illustrate views of the canopy of variety ‘K187’. FIG. 11G, FIG. 11H, and FIG. 11I illustrate views of flower buds on the tree of variety ‘K187’. FIG. 11J illustrates the tree trunk of variety ‘K187’.

FIG. 12A-FIG. 12G illustrate the plant or parts thereof of variety ‘K202’. FIG. 12A illustrate the whole tree of variety ‘K202’. FIG. 12B, FIG. 12C, and FIG. 12D illustrate views of the leaves and seed pods of variety ‘K202’. FIG. 12E illustrates the canopy of variety ‘K202’. FIG. 12F and FIG. 12G illustrate views of seed pods and seeds of variety ‘K202’.

FIG. 13A-FIG. 13E illustrate the plant or parts thereof of variety ‘K205’. FIG. 13A illustrate the whole tree of variety ‘K205’. FIG. 13B, FIG. 13C, and FIG. 13D illustrate views of the leaves and seed pods of variety ‘K205’. FIG. 13E illustrates the tree trunk of variety ‘K205’.

FIG. 14A-FIG. 14C illustrate the plant or parts thereof of variety ‘K206’. FIG. 14A and FIG. 14B illustrate views of the whole tree of variety ‘K206’. FIG. 14C illustrates the tree trunk of variety ‘K206’.

FIG. 15A-FIG. 15D illustrate the plant or parts thereof of variety ‘K207’. FIG. 15A illustrates the whole tree of variety ‘K207’. FIG. 15B and FIG. 15C illustrate views of the leaves and seed pods of variety ‘K207’. FIG. 15D illustrates the tree trunk of variety ‘K207’.

FIG. 16A-FIG. 16B illustrate the plant or parts thereof of variety ‘K208’. FIG. 16A illustrates the whole tree of variety ‘K208’. FIG. 16B illustrates the tree trunk of variety ‘K208’.

FIG. 17 illustrates the whole tree of variety ‘K209’.

FIG. 18A-FIG. 18D illustrate the plant or parts thereof of variety ‘K211’. FIG. 18A and FIG. 18B illustrate views of the whole tree of variety ‘K211’. FIG. 18C illustrates the canopy of variety ‘K211’. FIG. 18D illustrates the tree trunk of variety ‘K211’.

FIG. 19A-FIG. 19H illustrate the plant or parts thereof of variety ‘K219’. FIG. 19A and FIG. 19B illustrate views of the whole tree of variety ‘K219’. FIG. 19C and FIG. 19D illustrate views of the canopy of variety ‘K219’. FIG. 19E and FIG. 19F illustrate views of the tree trunk of variety ‘K219’. FIG. 19G and FIG. 19H illustrate views of seed pods and seeds of variety ‘K219’.

FIG. 20A-FIG. 20D illustrate the plant or parts thereof of variety ‘K268’. FIG. 20A illustrates the whole tree of variety ‘K268’. FIG. 20B and FIG. 20C illustrate views of the canopy of variety ‘K268’. FIG. 20D illustrates the tree trunk of variety ‘K268’.

FIG. 21A-FIG. 21B illustrate the plant or parts thereof of variety ‘K303’. FIG. 21A illustrates the whole tree of variety ‘K303’. FIG. 21B illustrates the tree trunk of variety ‘K303’.

FIG. 22A-FIG. 22B illustrate the plant or parts thereof of variety ‘K309’. FIG. 22A illustrates the whole tree of variety ‘K309’. FIG. 22B illustrates the tree trunk of variety ‘K309’.

FIG. 23 illustrates the whole tree of variety ‘K605’.

FIG. 24A-FIG. 24C illustrate the plant or parts thereof of variety ‘K607’. FIG. 24A and FIG. 24B illustrate views of the whole tree of variety ‘K607’. FIG. 24C illustrates the tree trunk of variety ‘K607’.

FIG. 25A-FIG. 25C illustrate the plant or parts thereof of variety ‘K608’. FIG. 25A illustrates the whole tree of variety ‘K608’. FIG. 25B and FIG. 25C illustrate views of the canopy of variety ‘K608’.

FIG. 26A-FIG. 26B illustrate the plant or parts thereof of variety ‘K609’. FIG. 26A illustrates the whole tree of variety ‘K609’. FIG. 26B illustrates the tree trunk of variety ‘K609’.

FIG. 27A-FIG. 27C illustrate the plant or parts thereof of variety ‘K610’. FIG. 27A illustrates the whole tree of variety ‘K610’. FIG. 27B illustrates the tree trunk of variety ‘K610’. FIG. 27C illustrates a seed pod and seeds of variety ‘K610’.

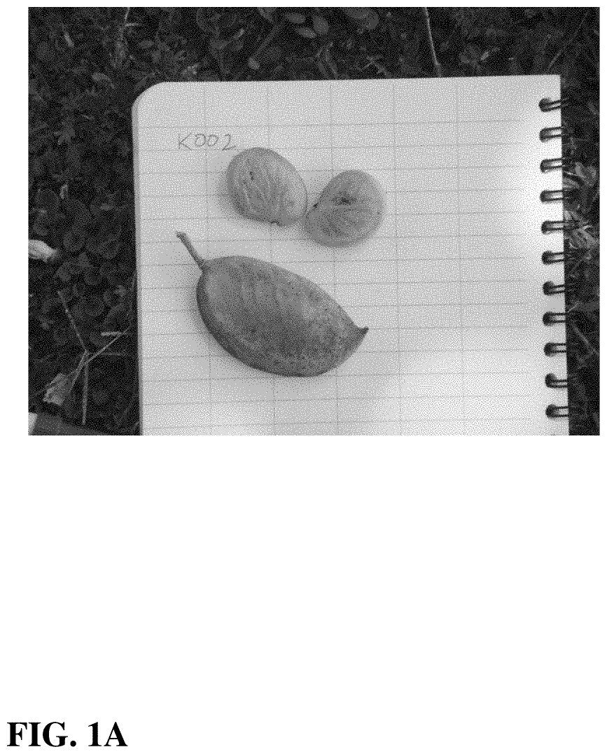

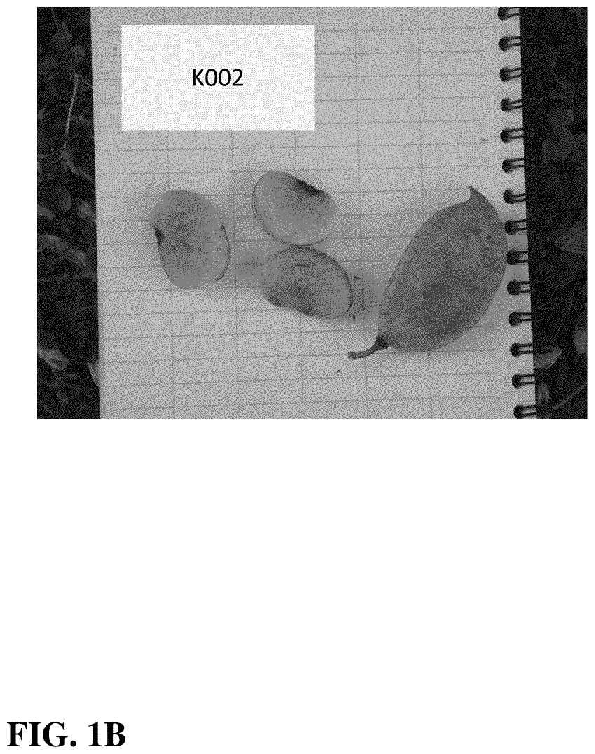

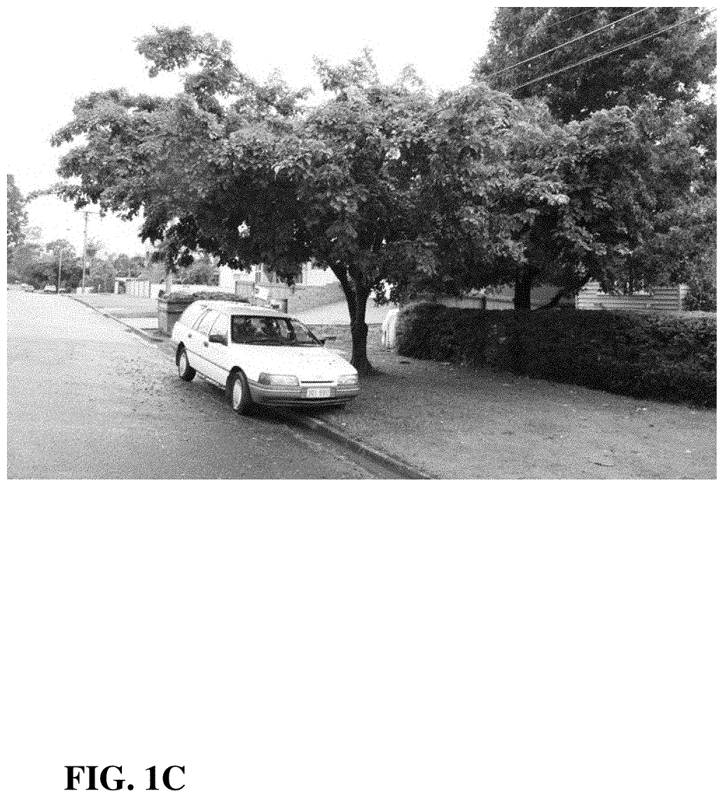

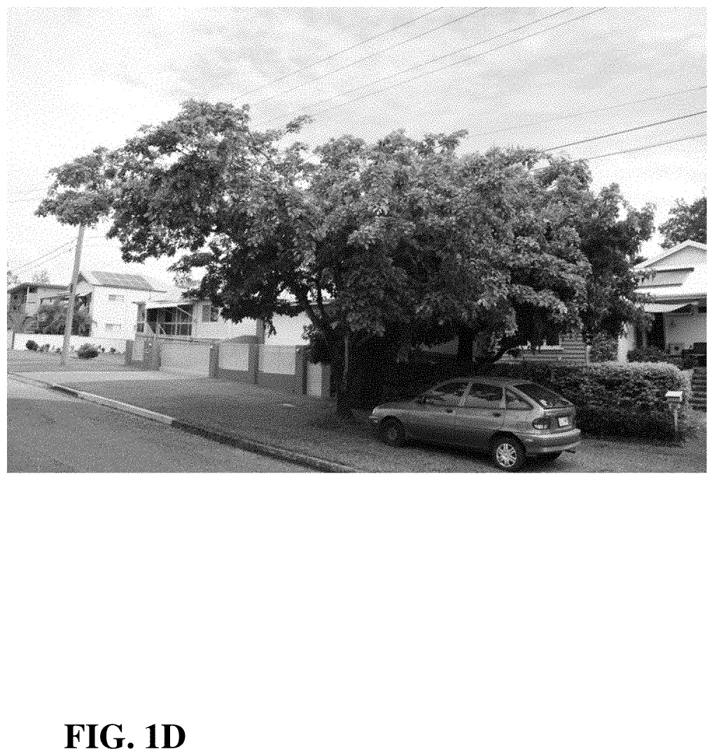

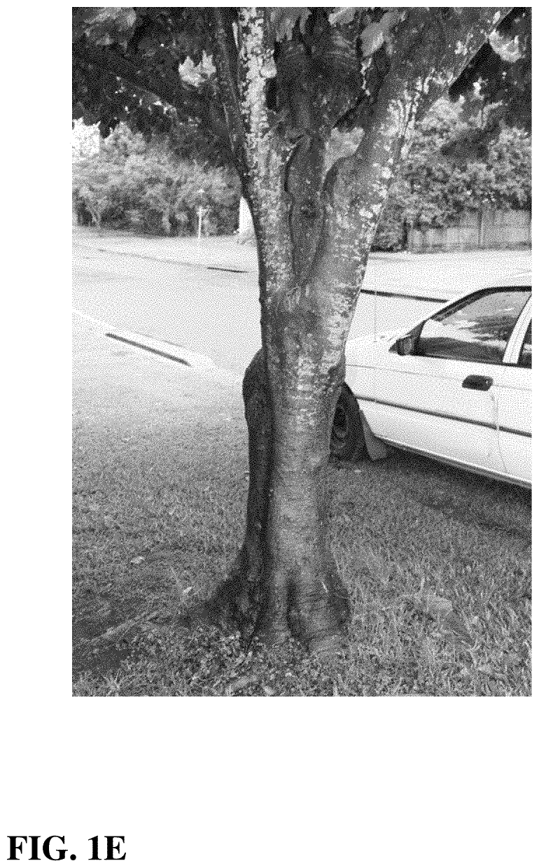

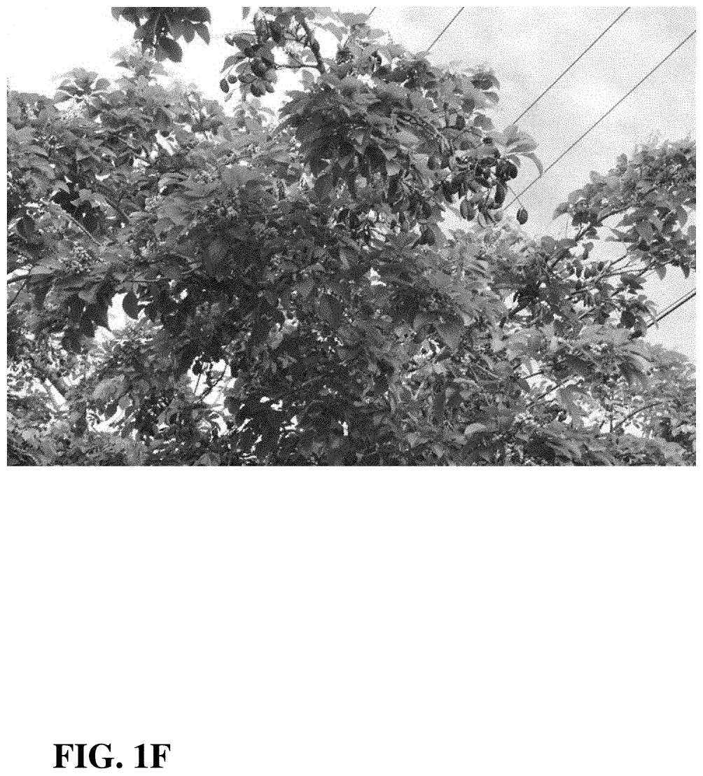

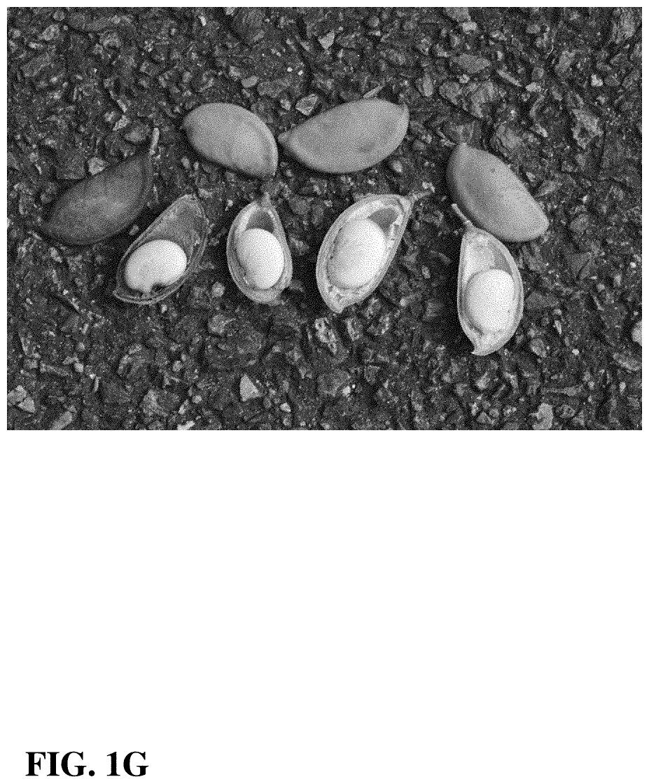

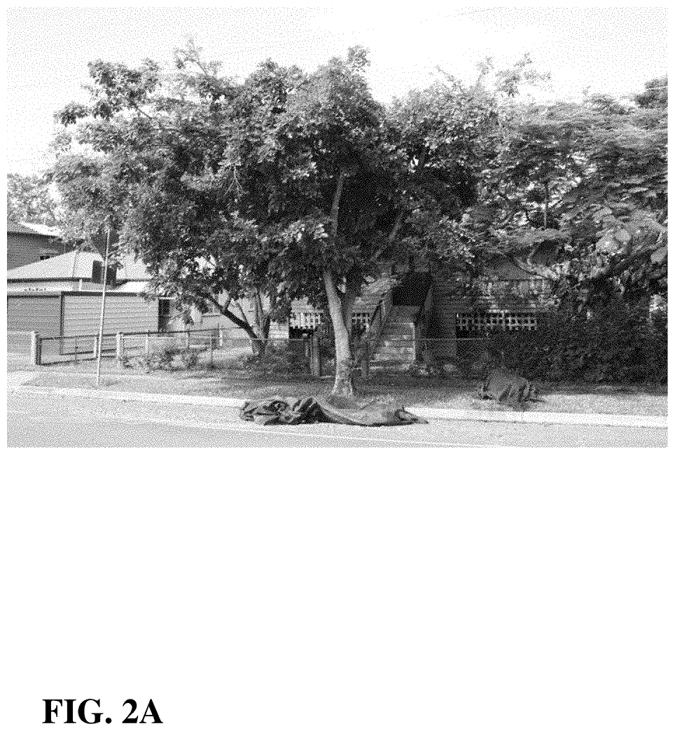

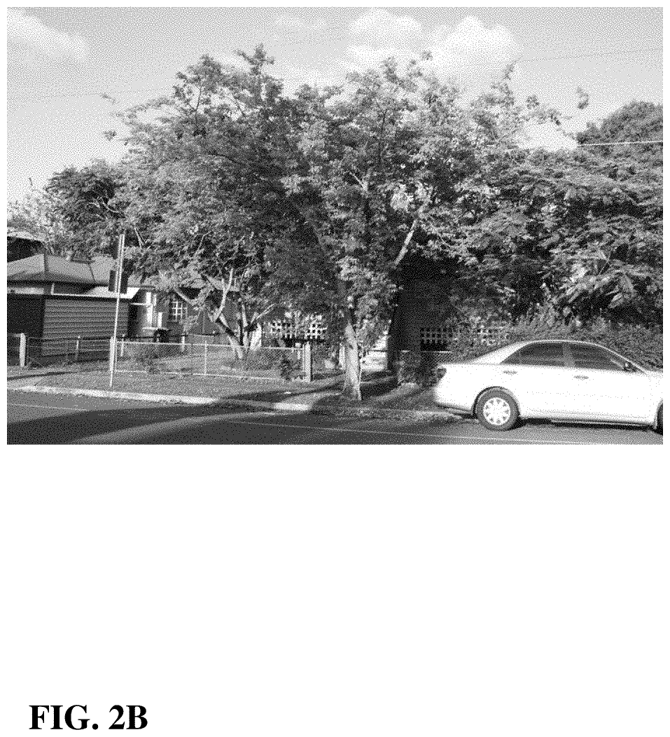

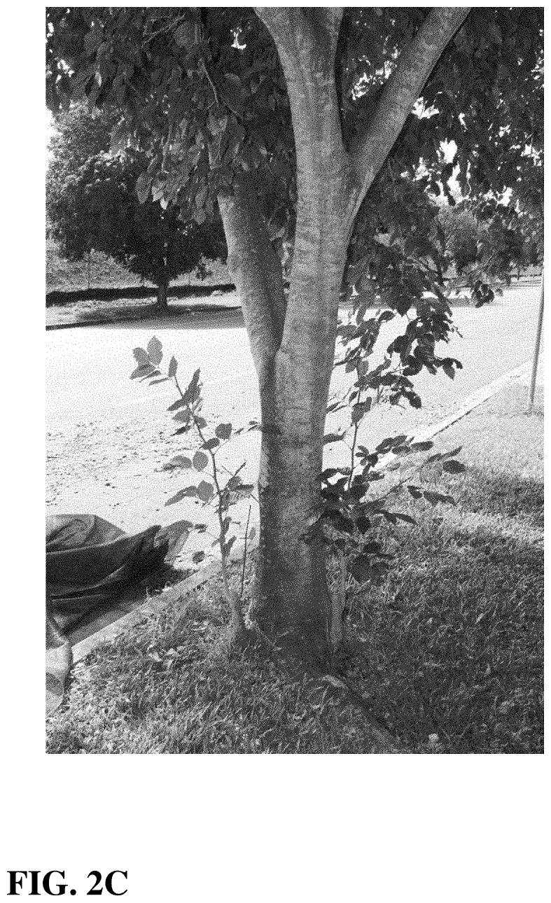

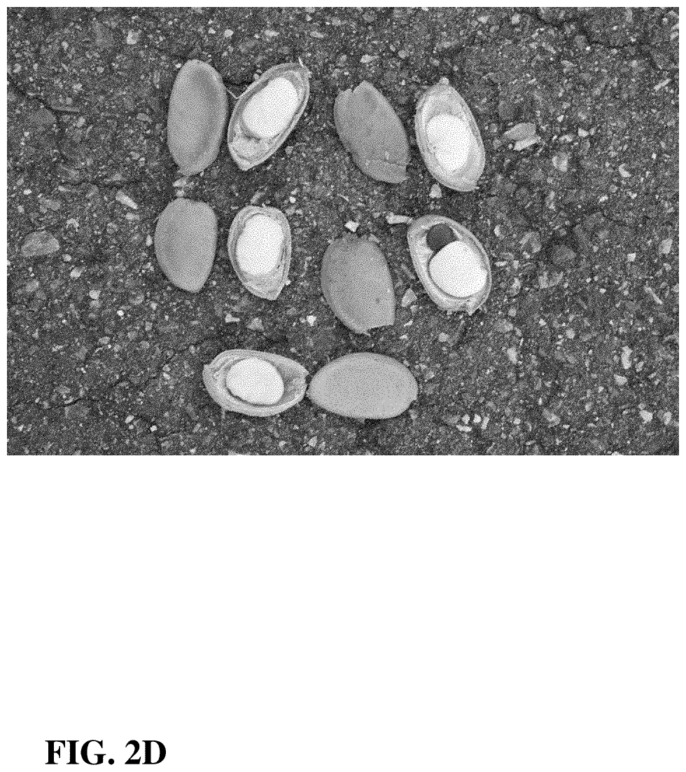

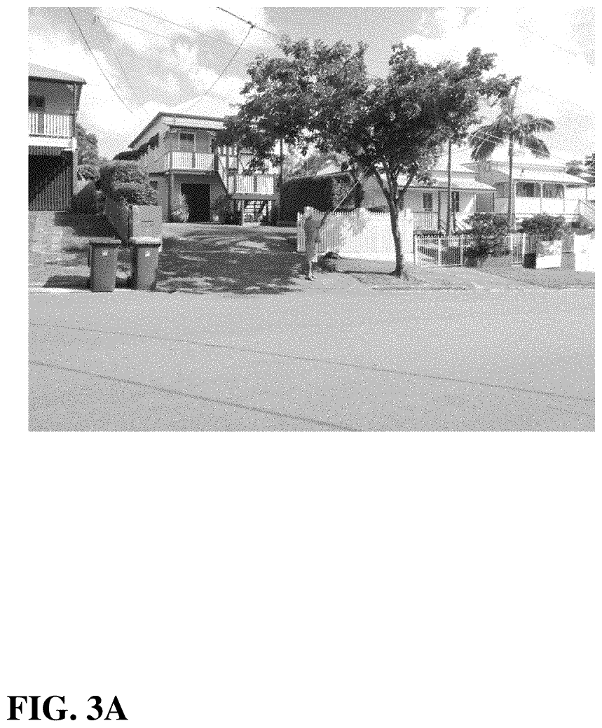

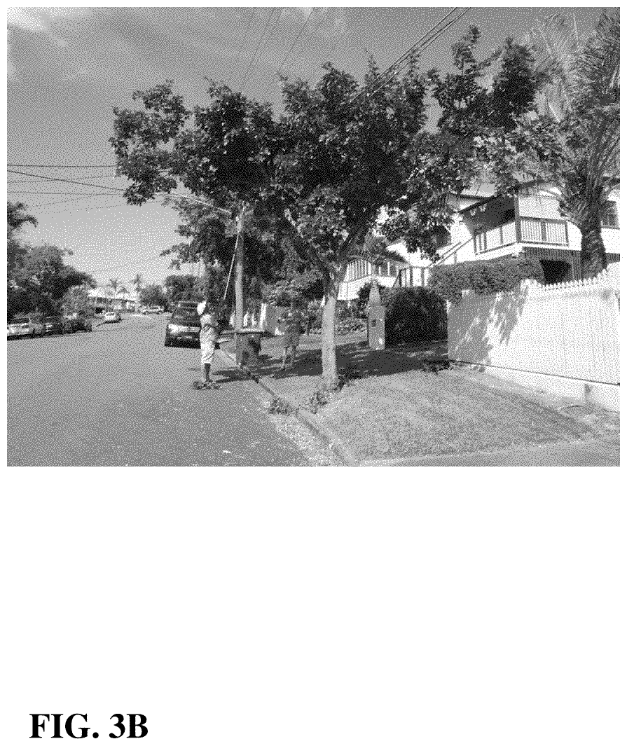

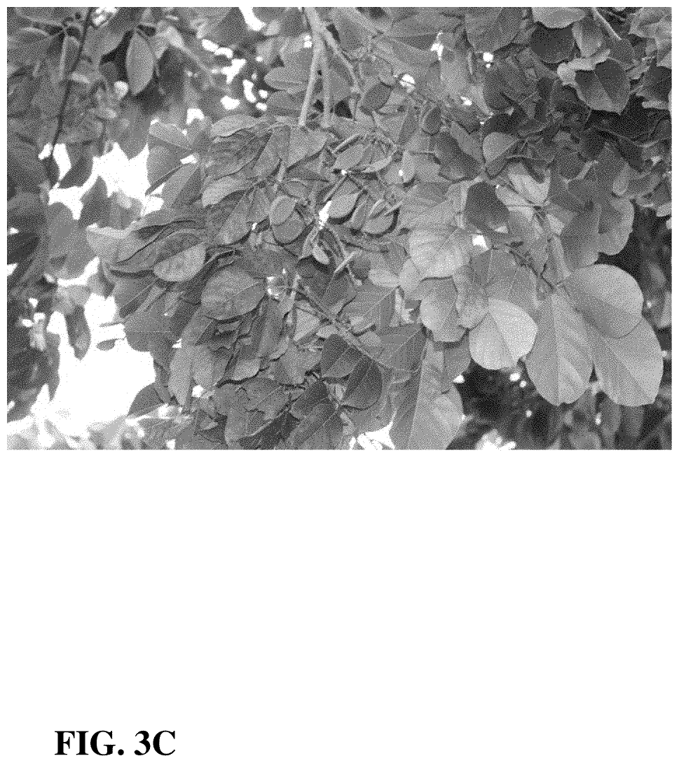

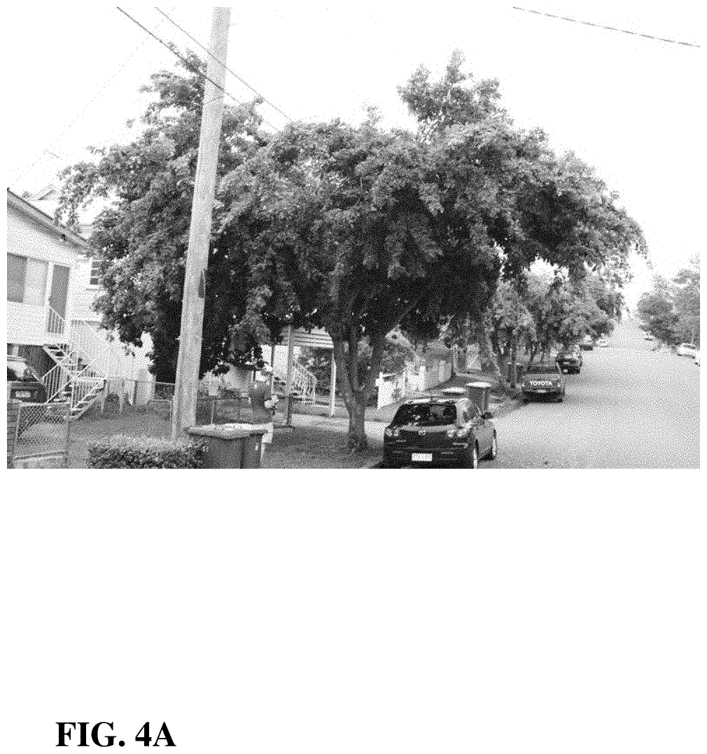

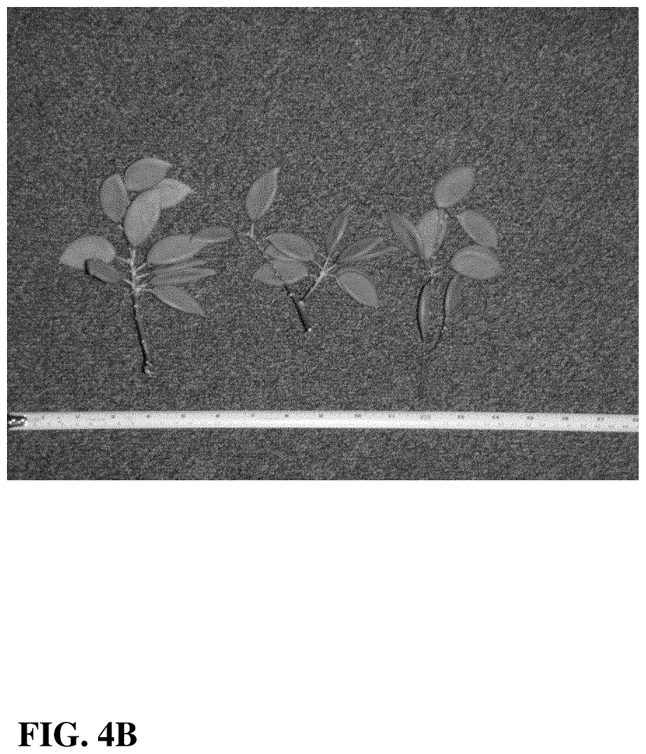

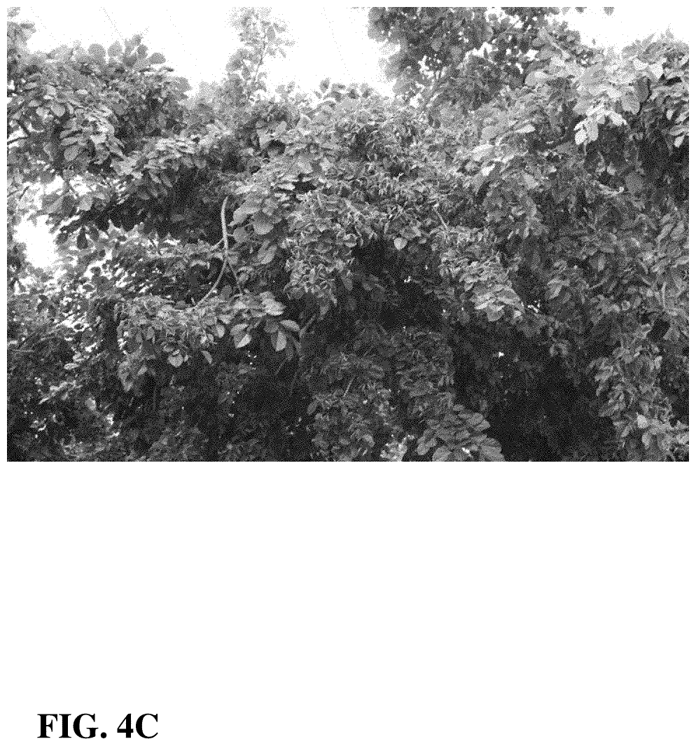

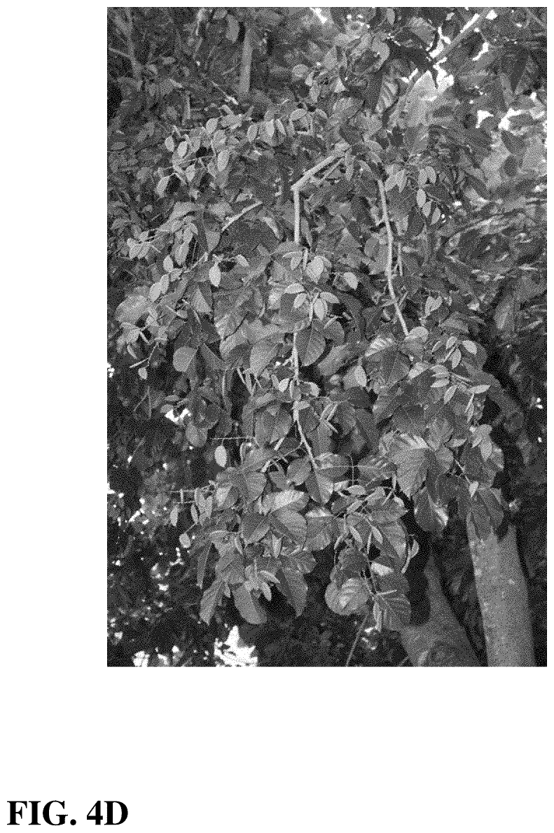

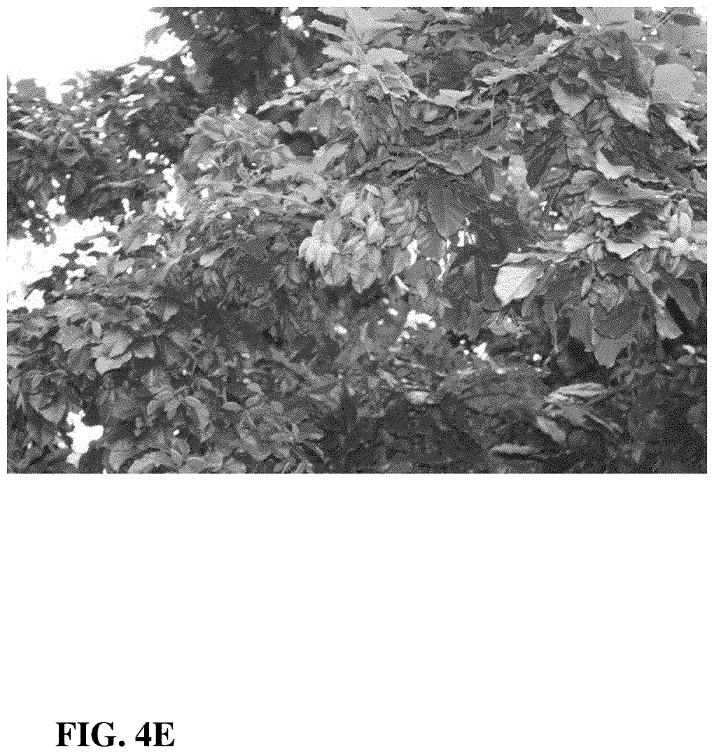

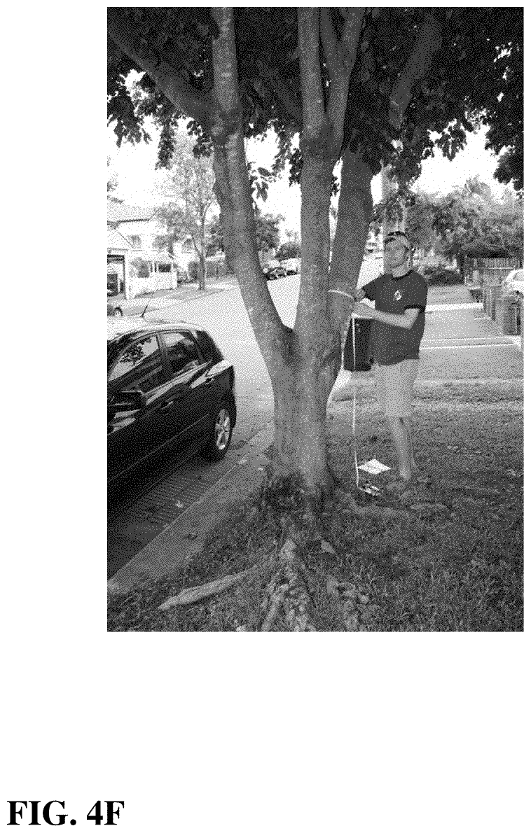

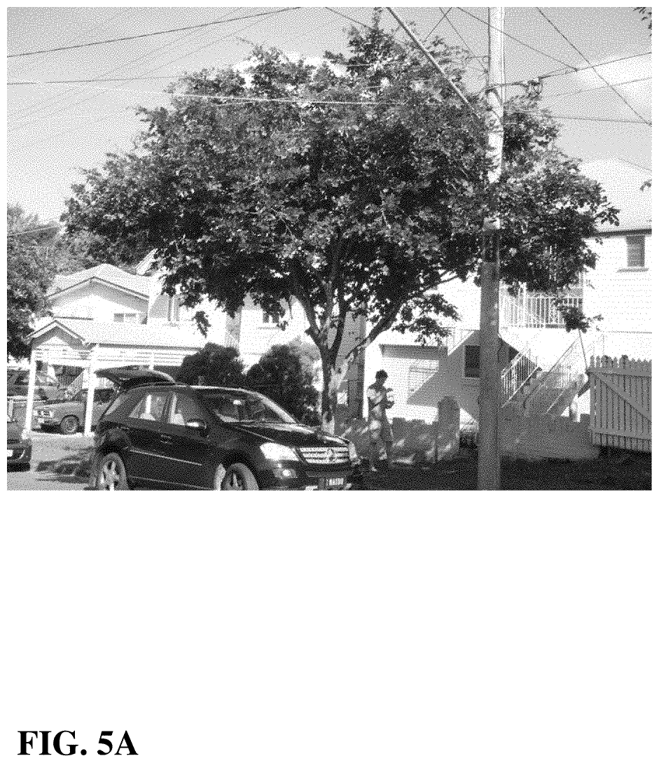

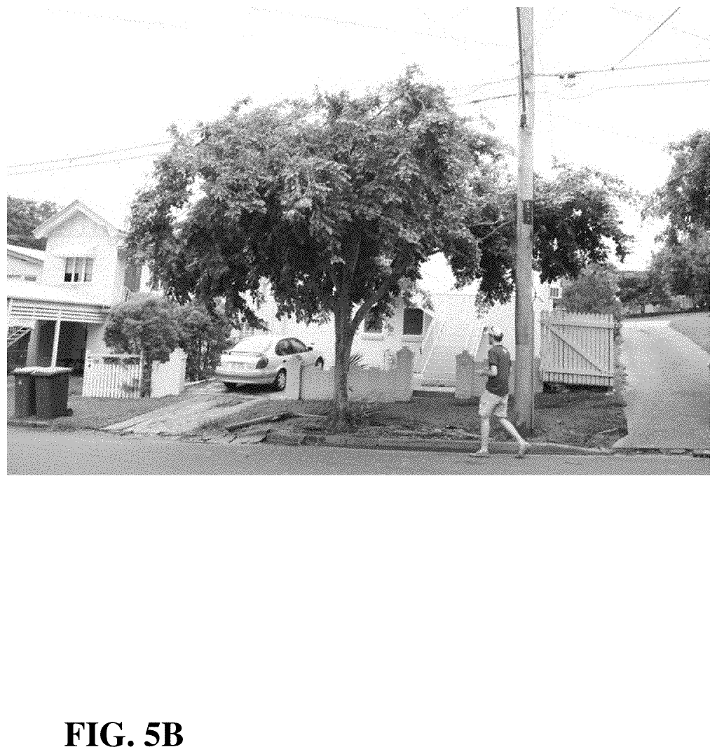

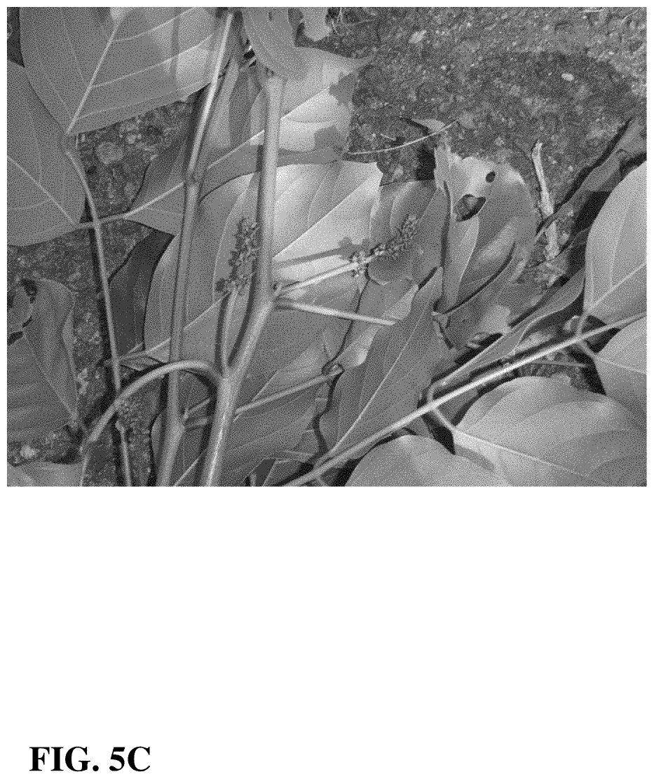

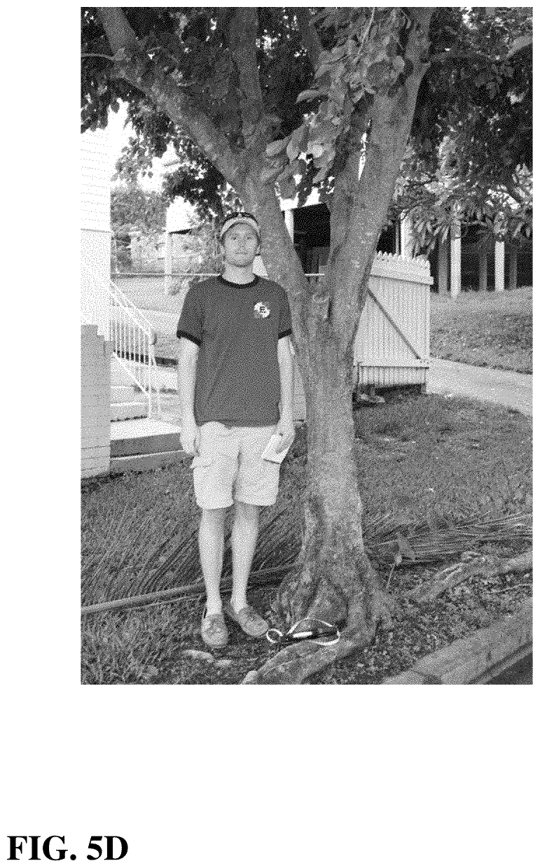

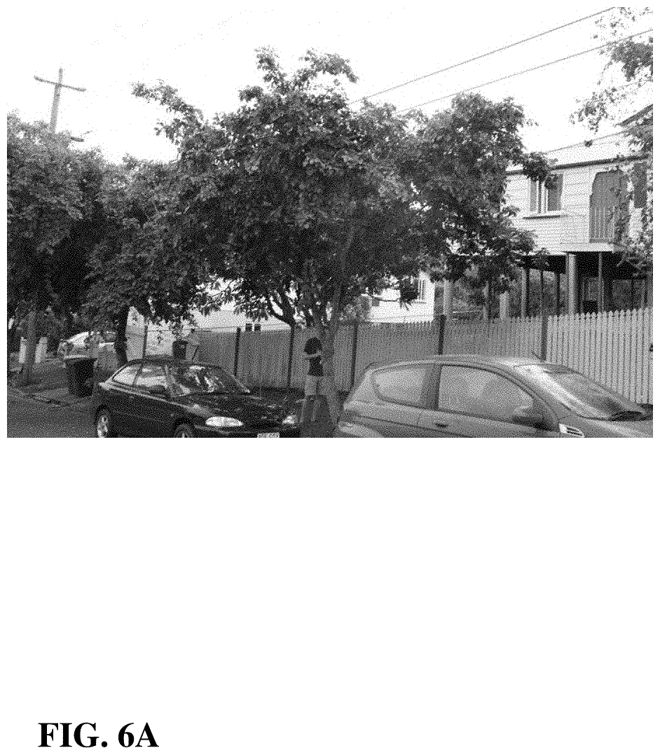

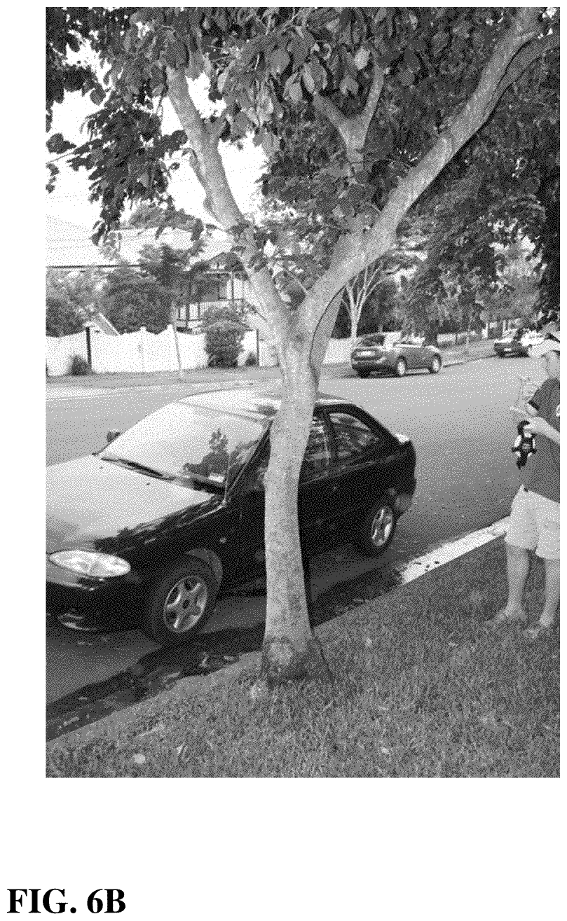

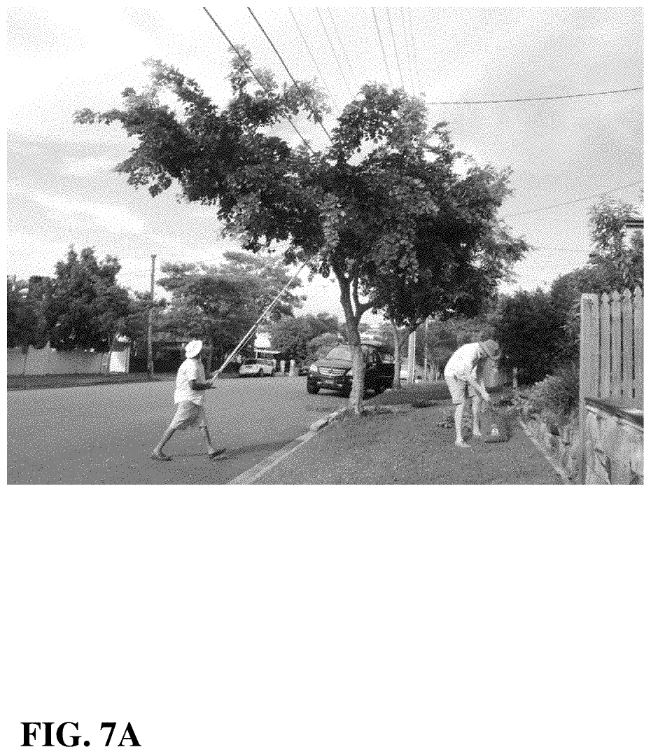

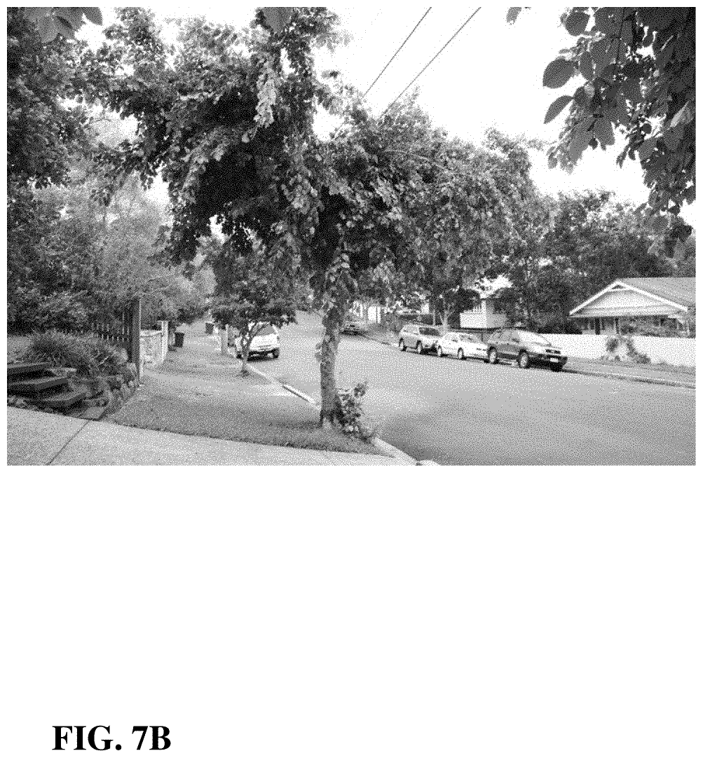

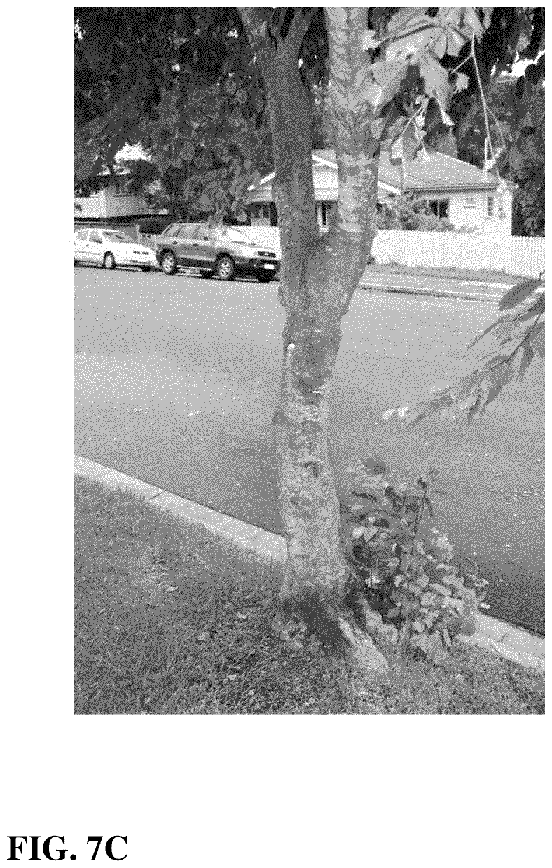

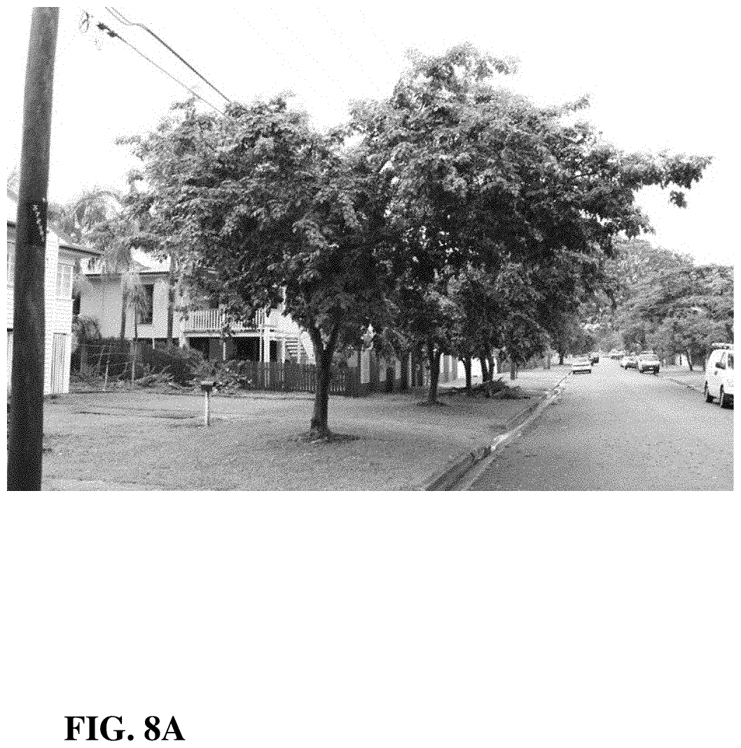

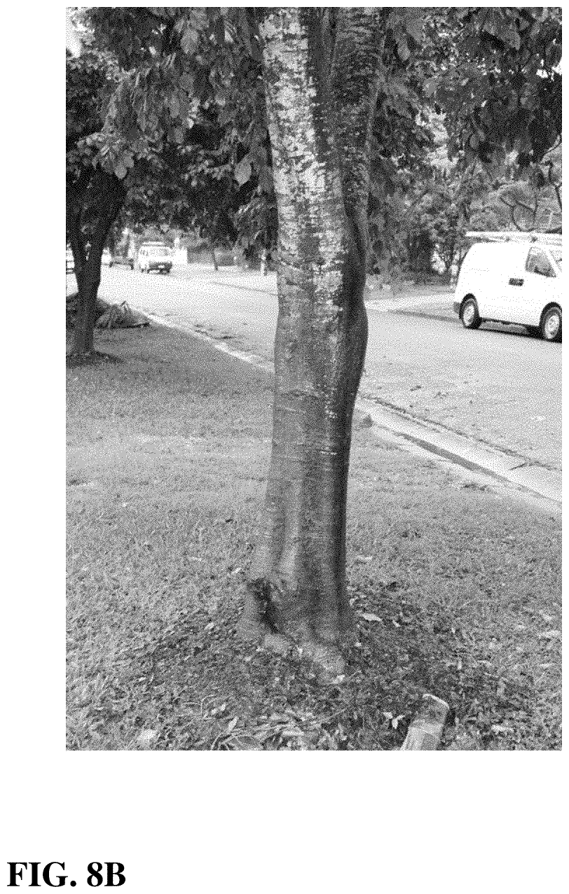

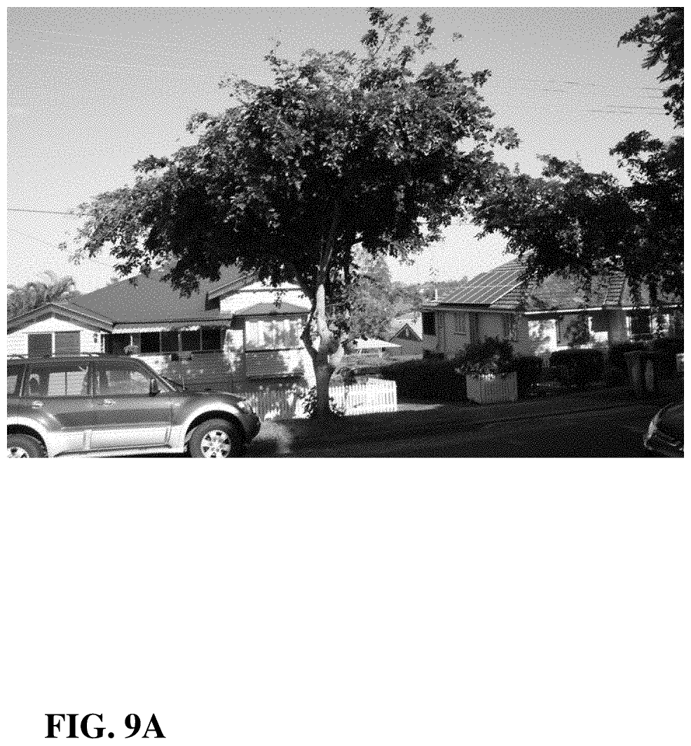

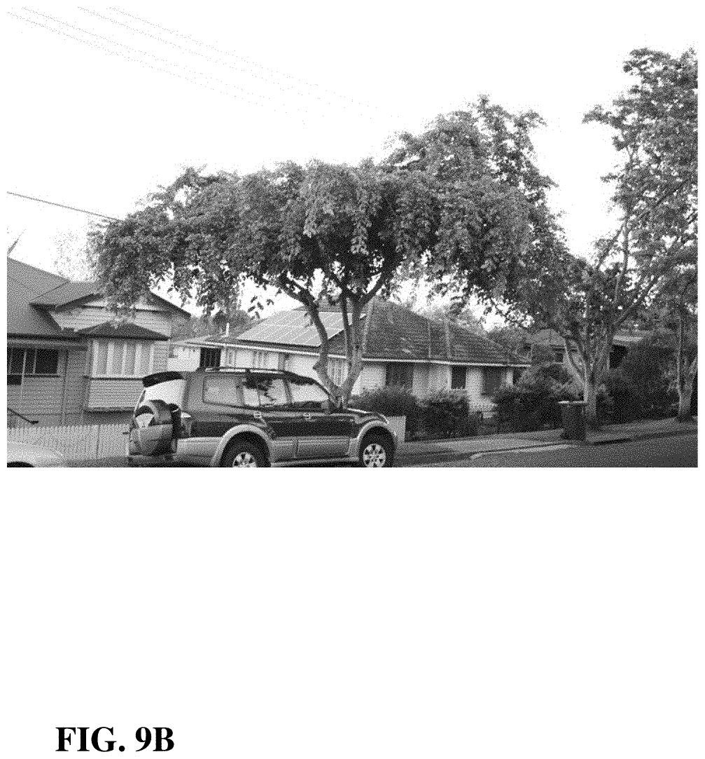

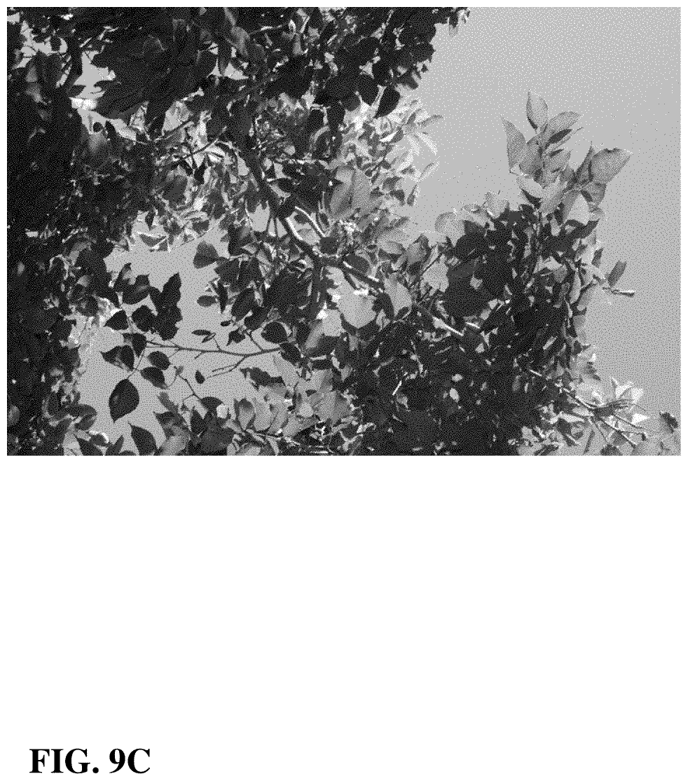

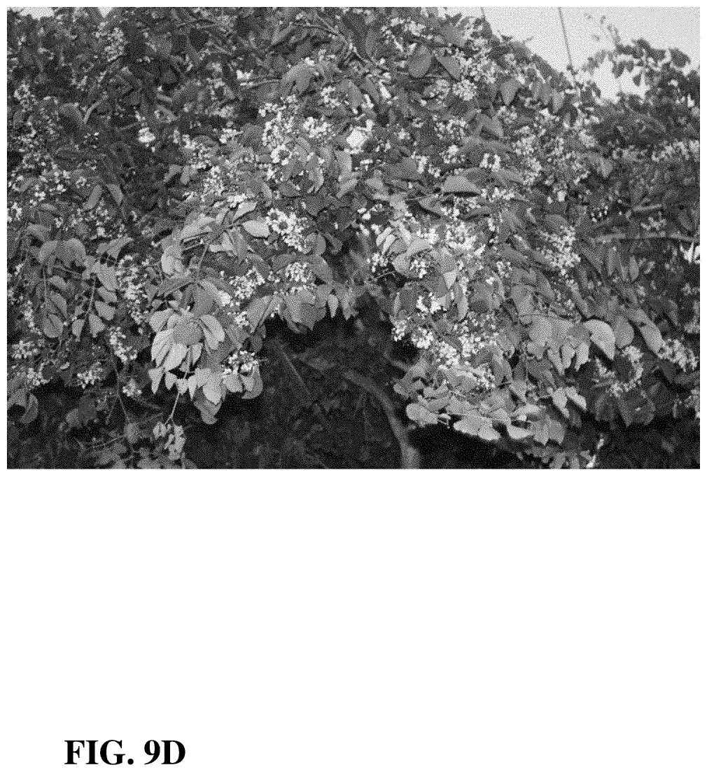

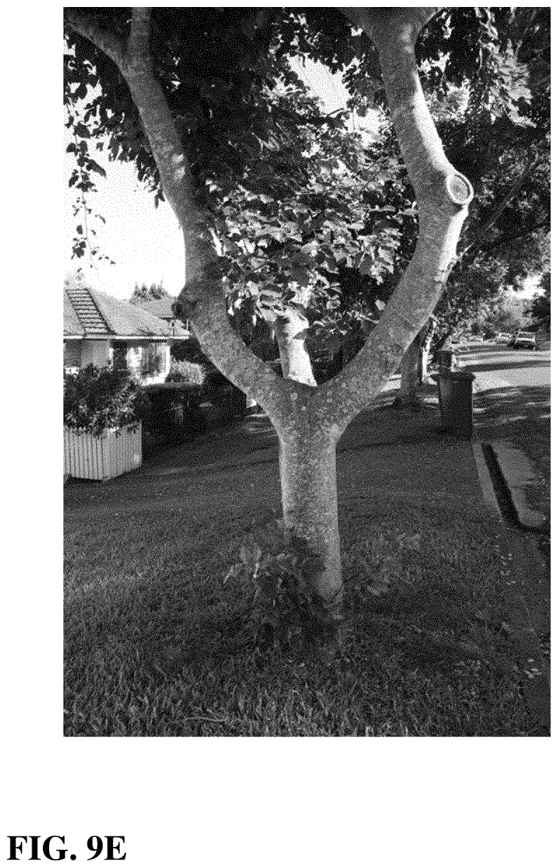

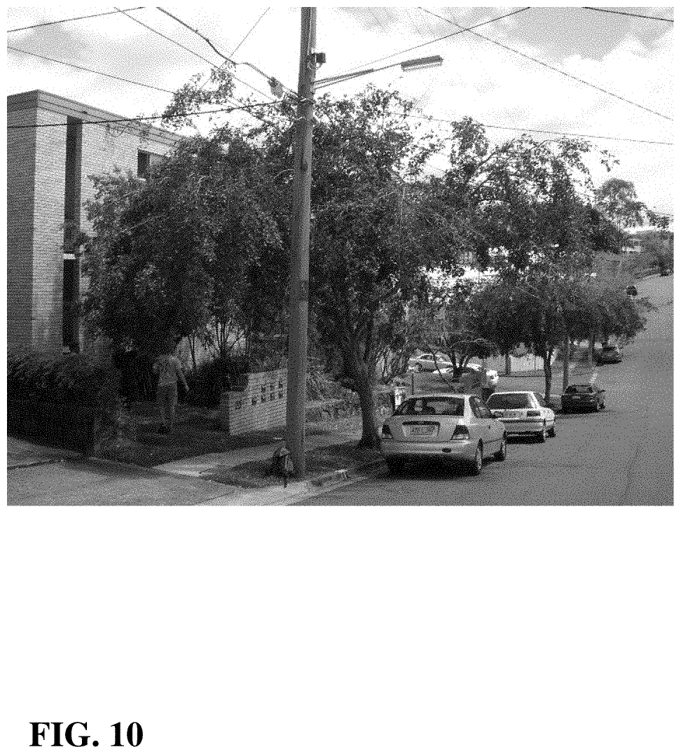

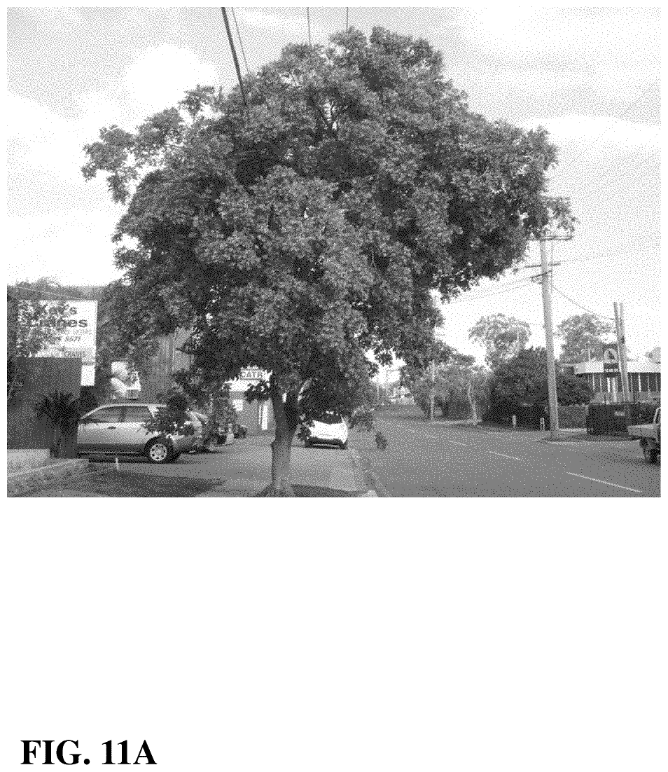

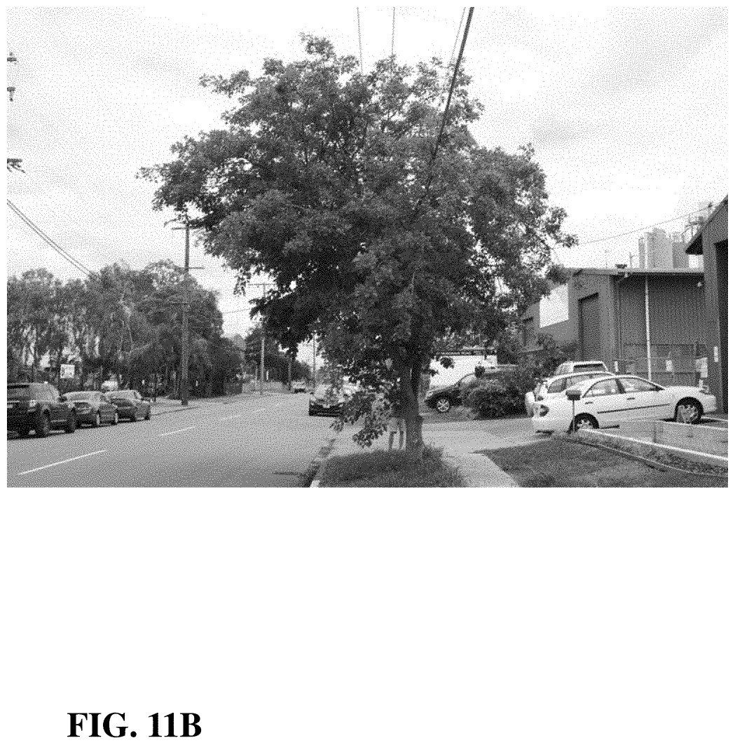

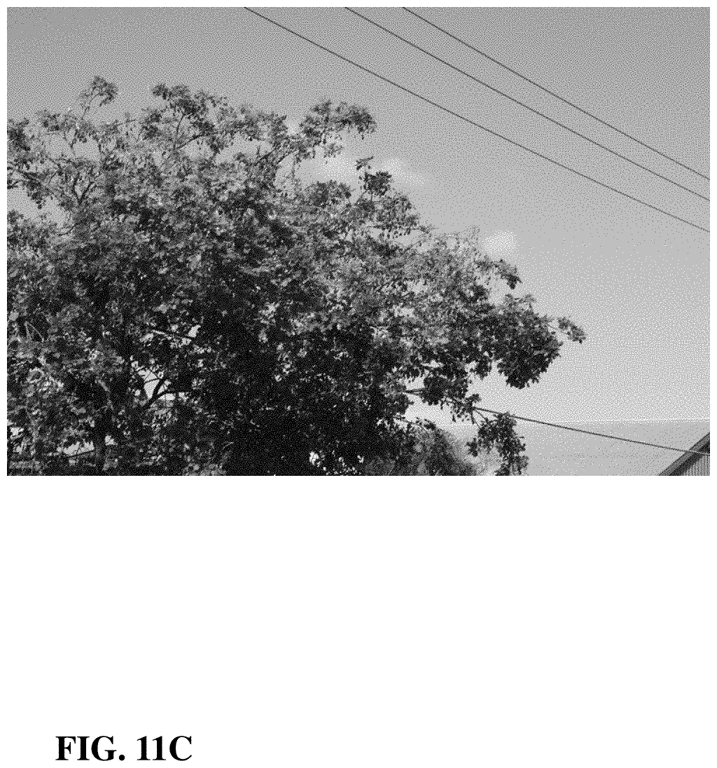

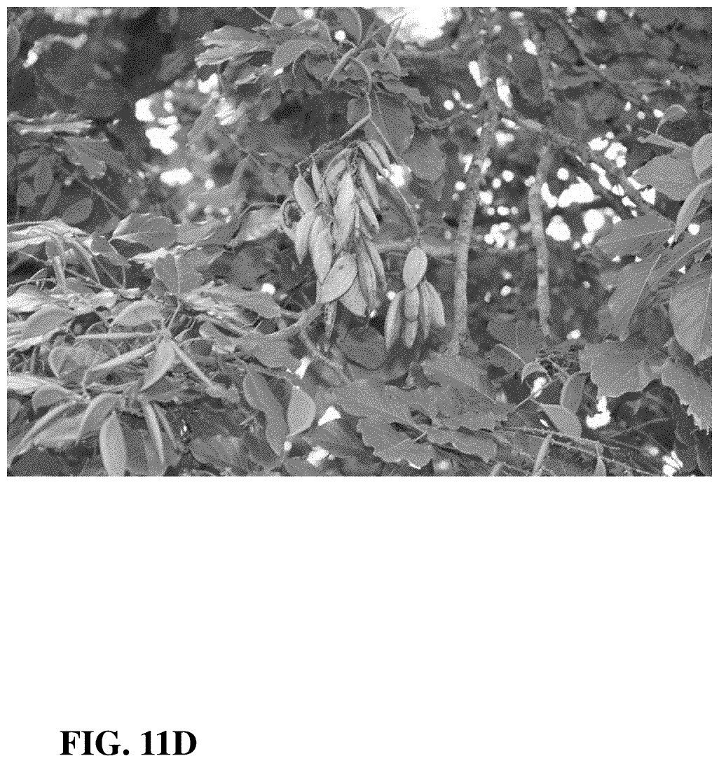

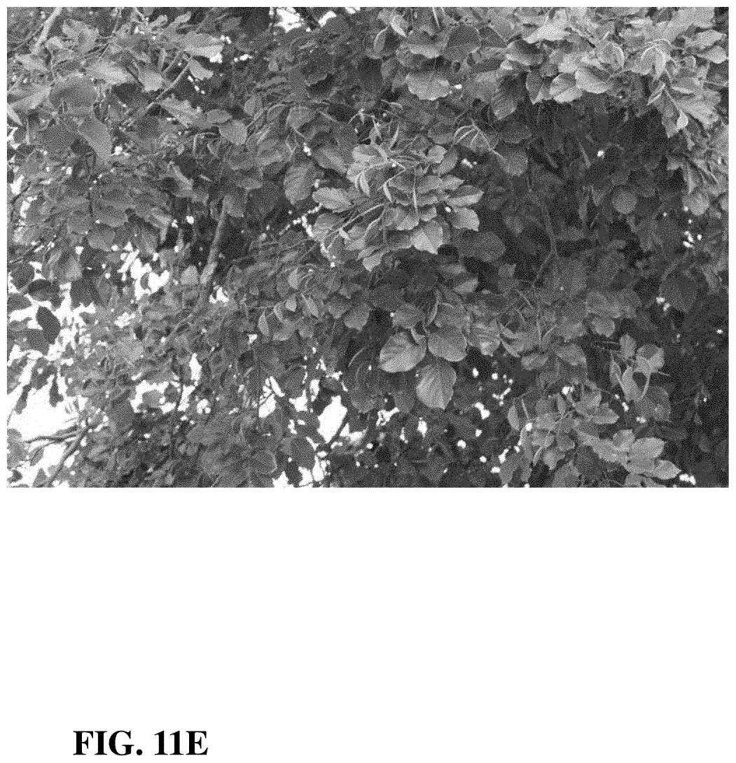

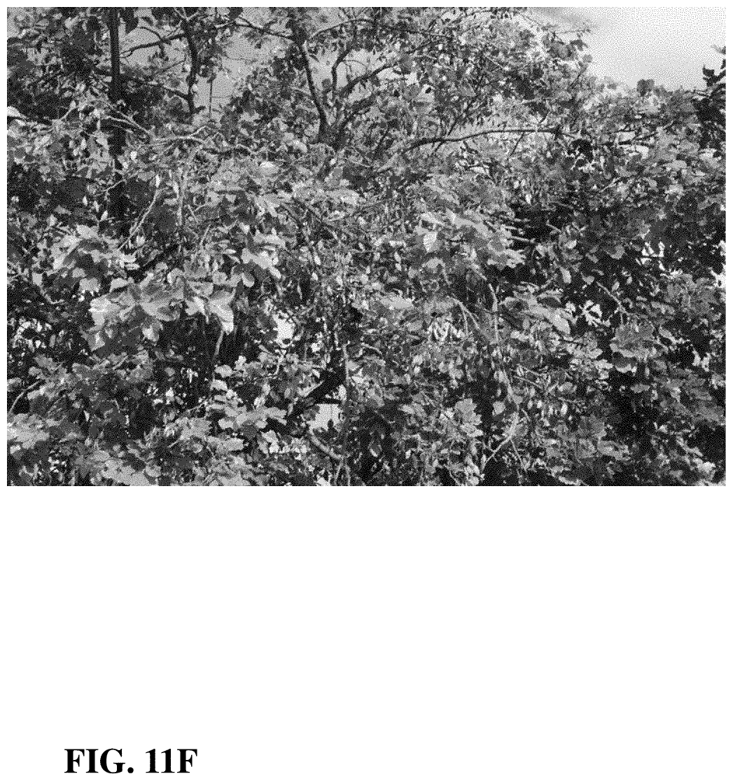

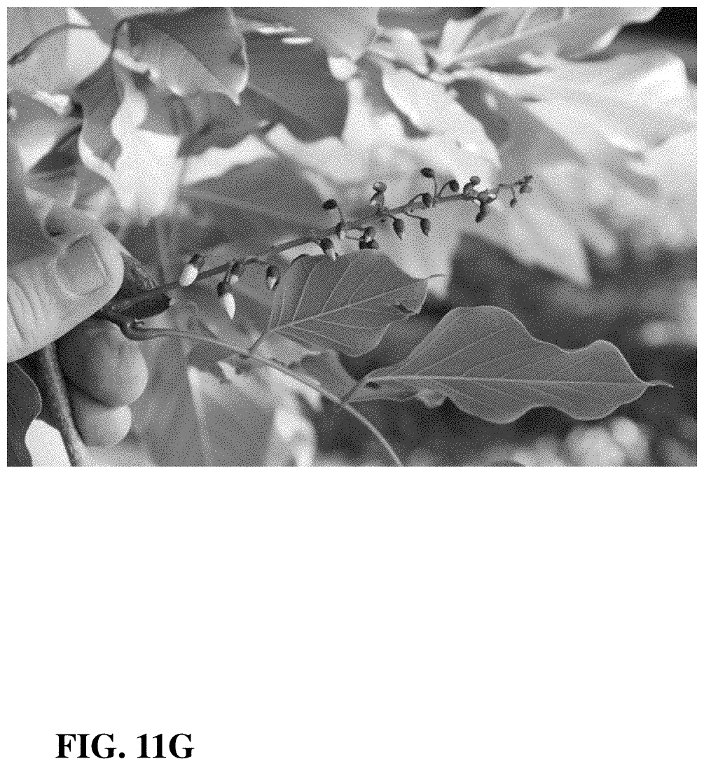

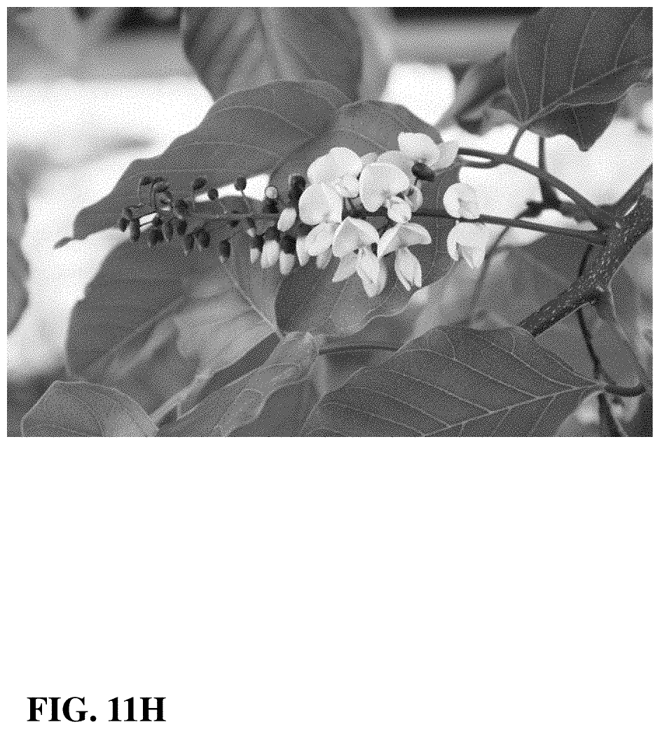

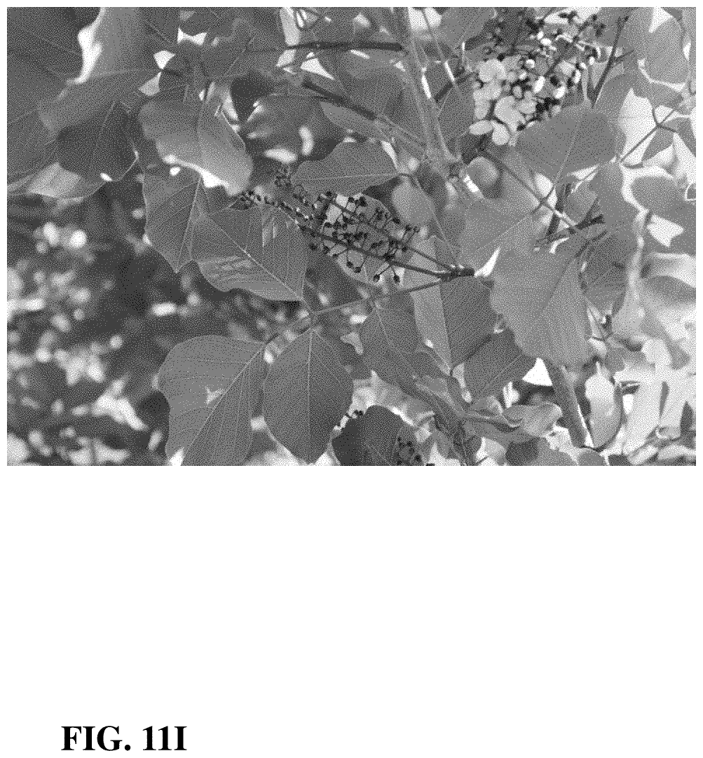

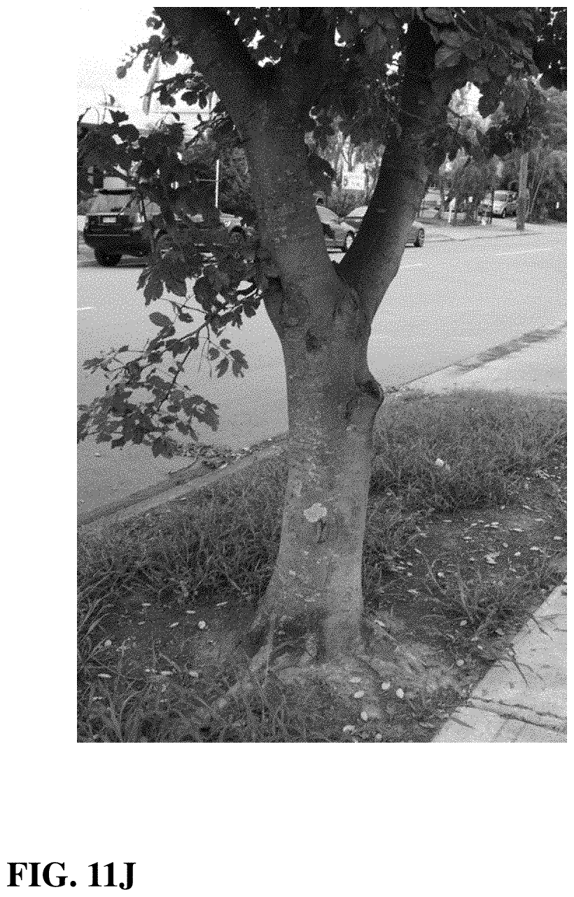

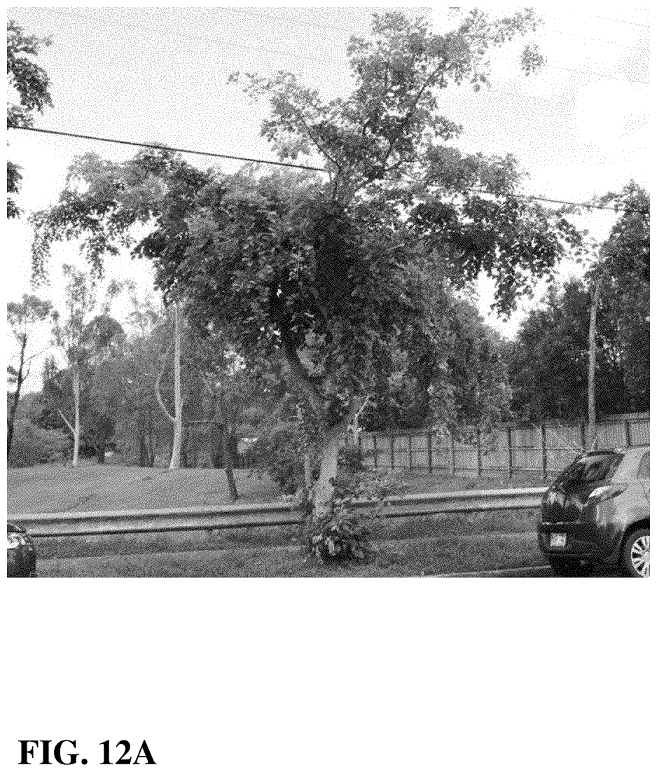

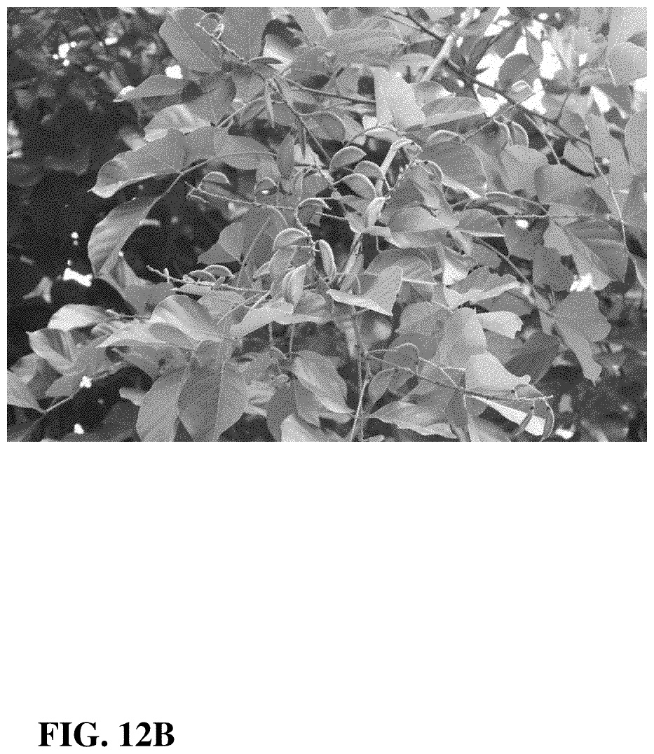

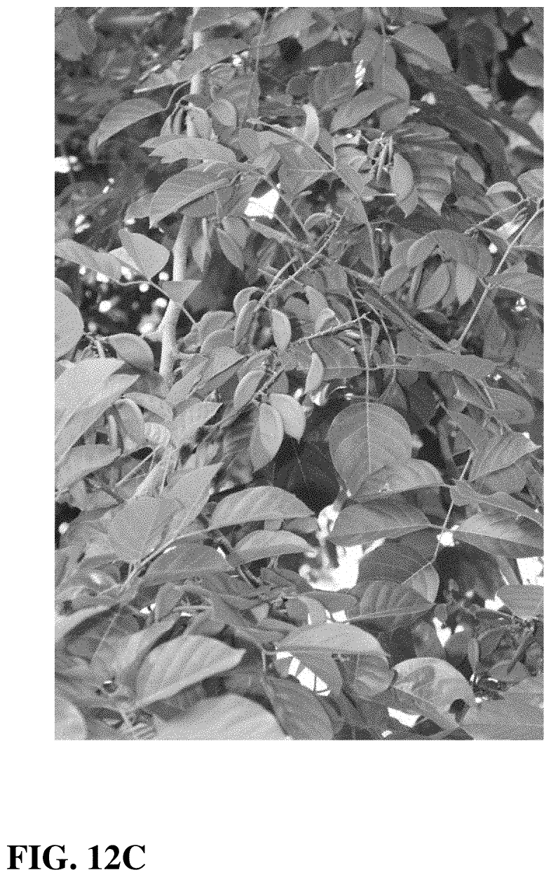

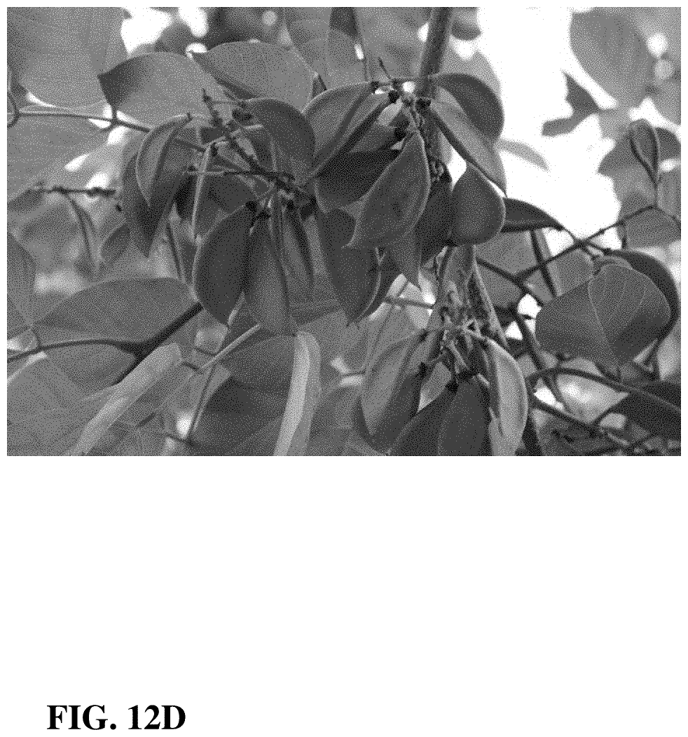

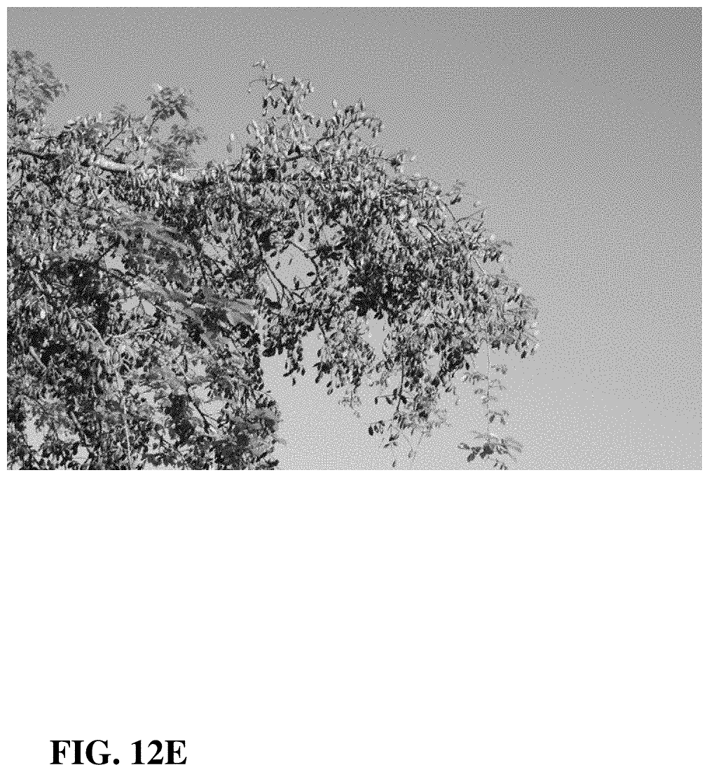

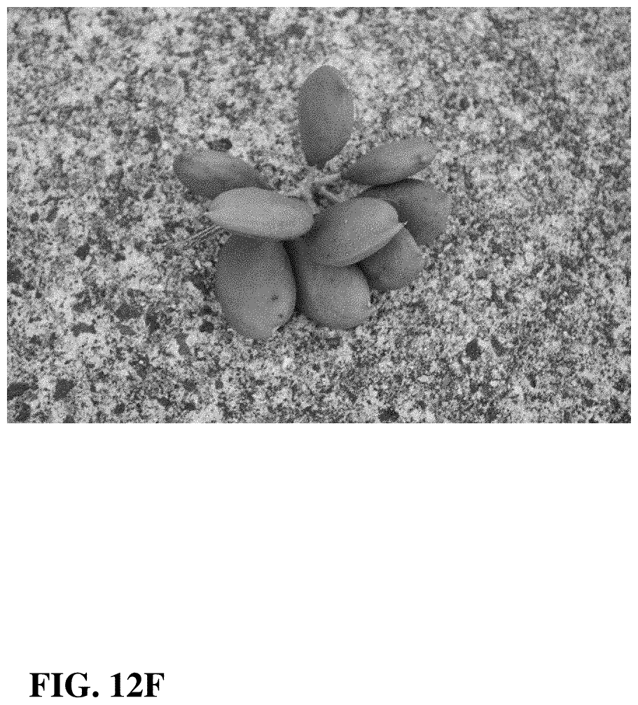

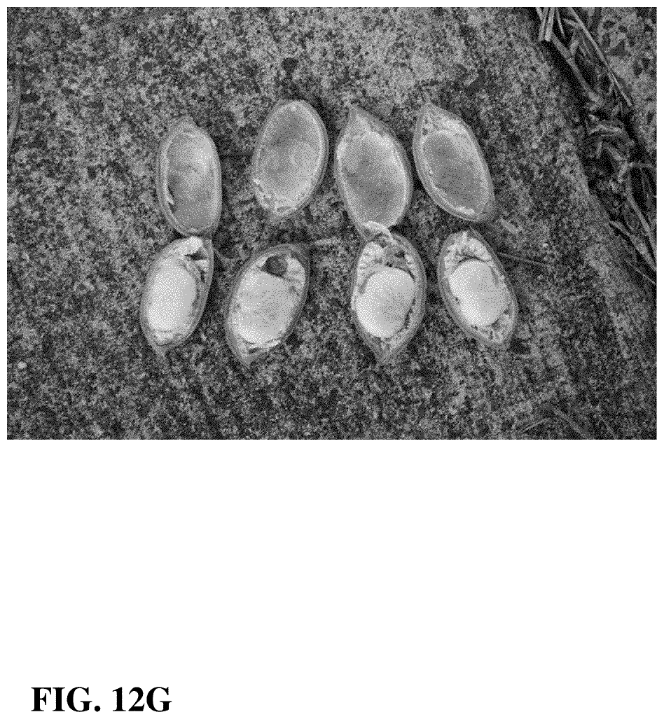

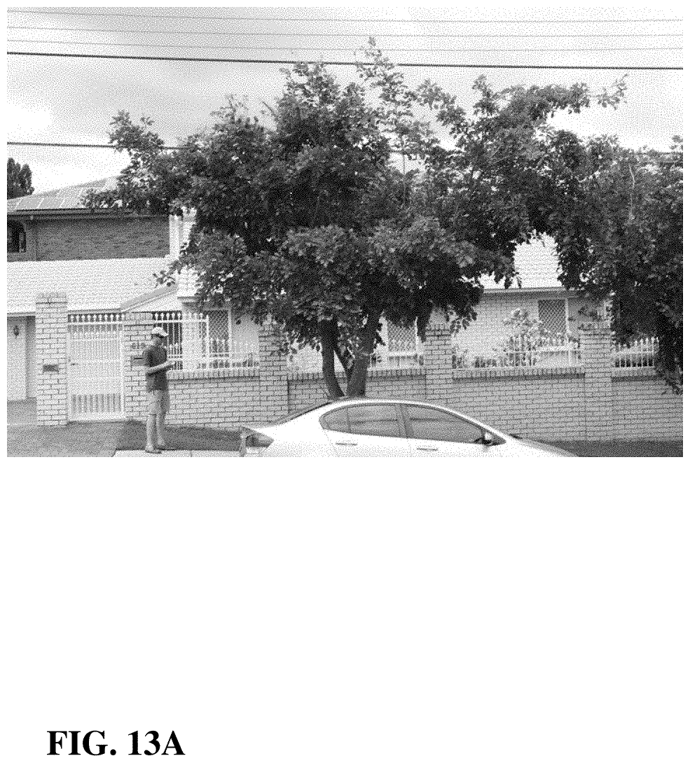

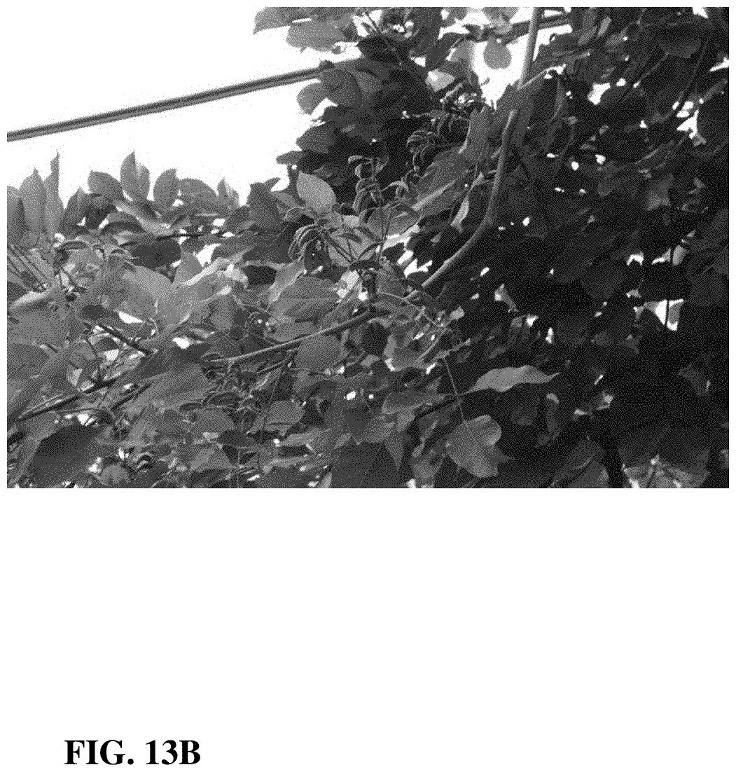

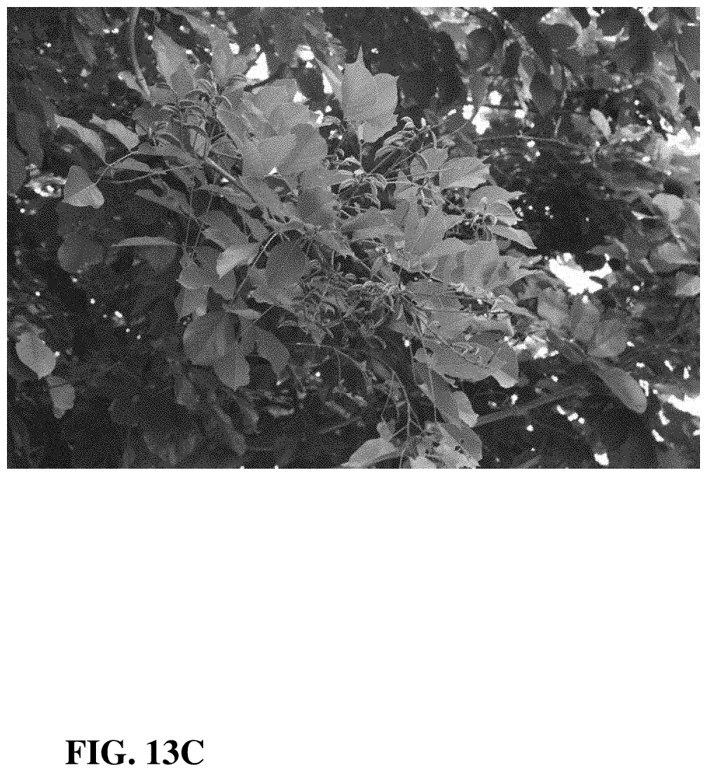

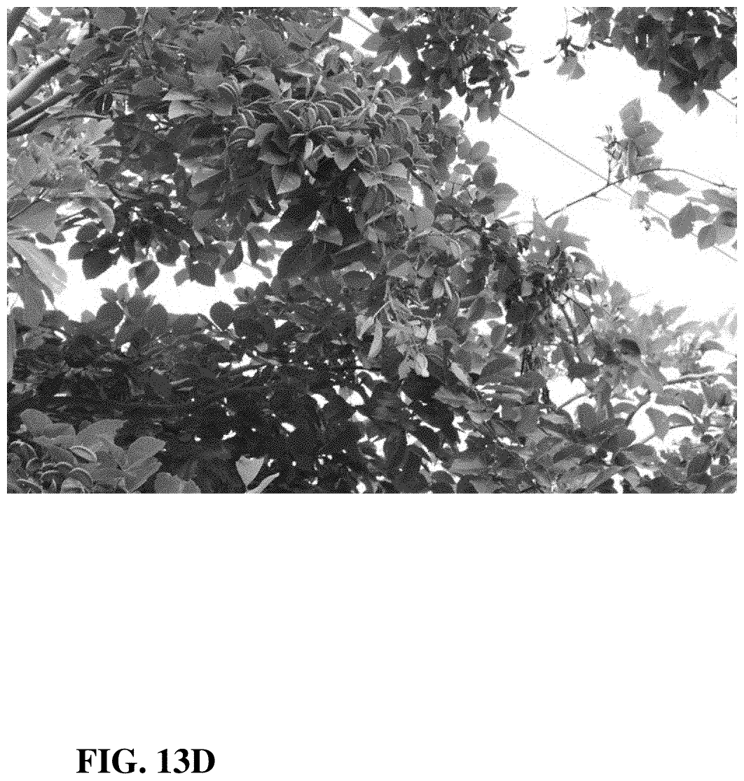

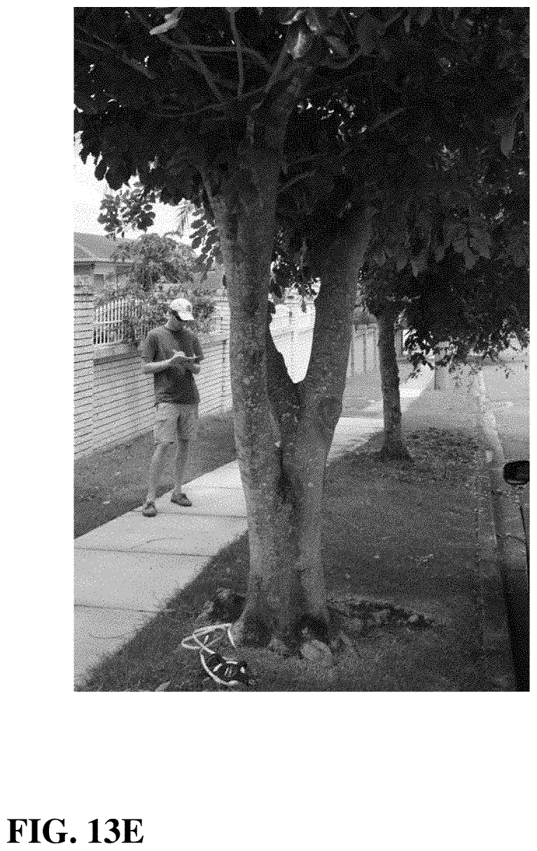

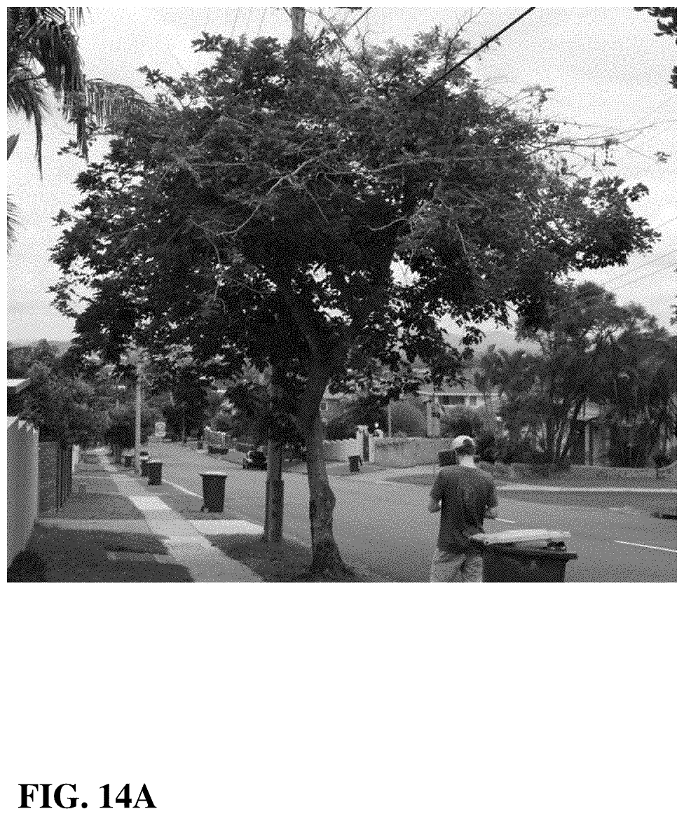

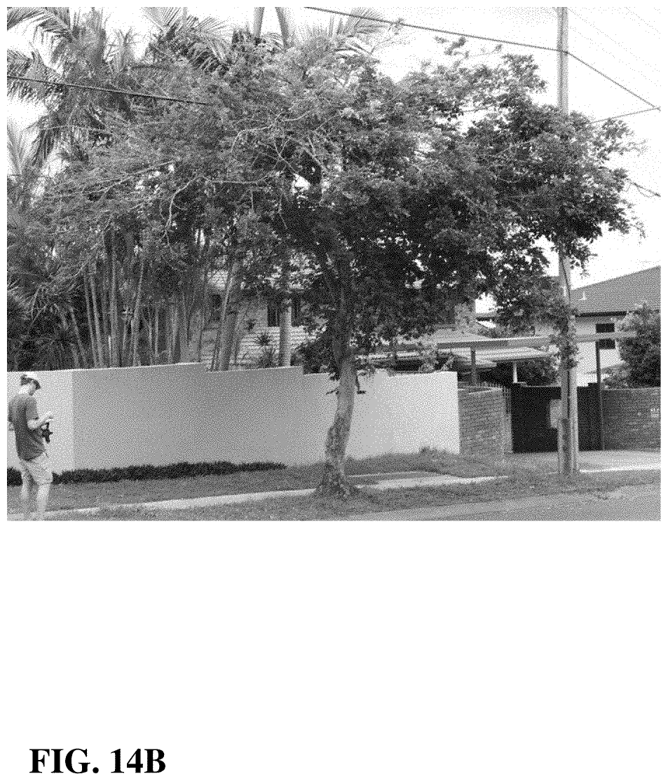

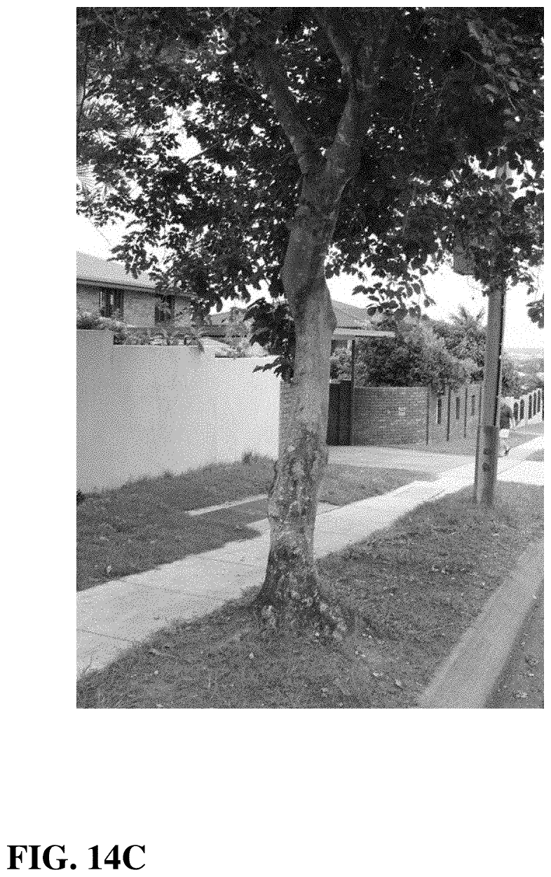

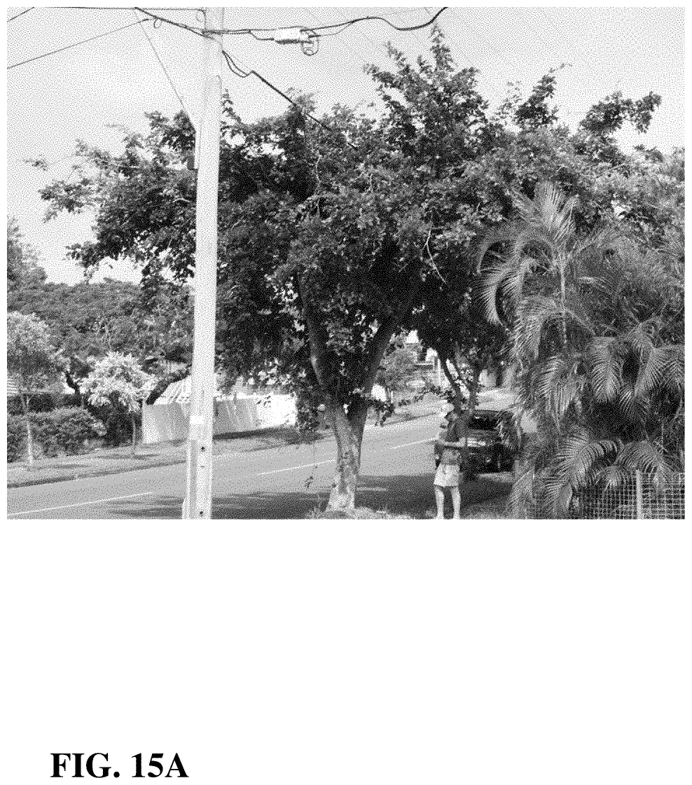

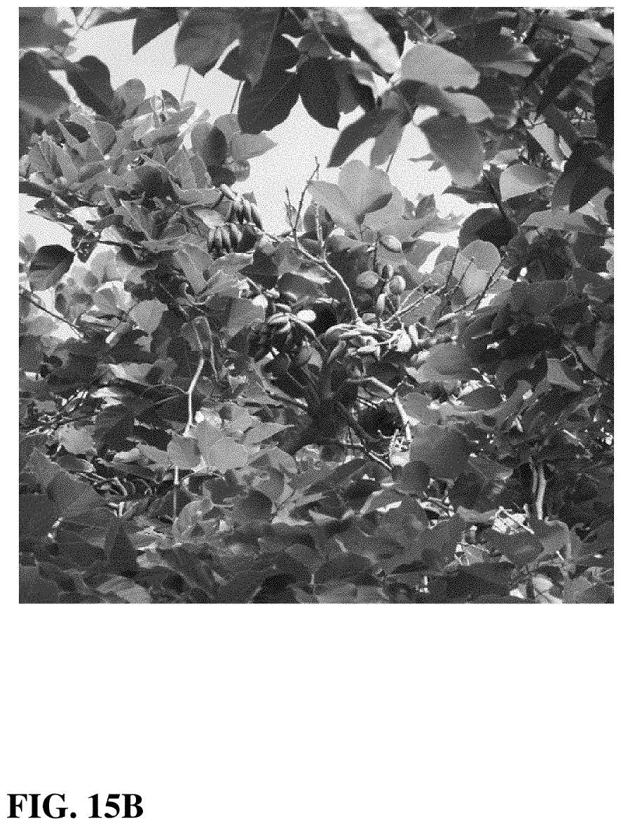

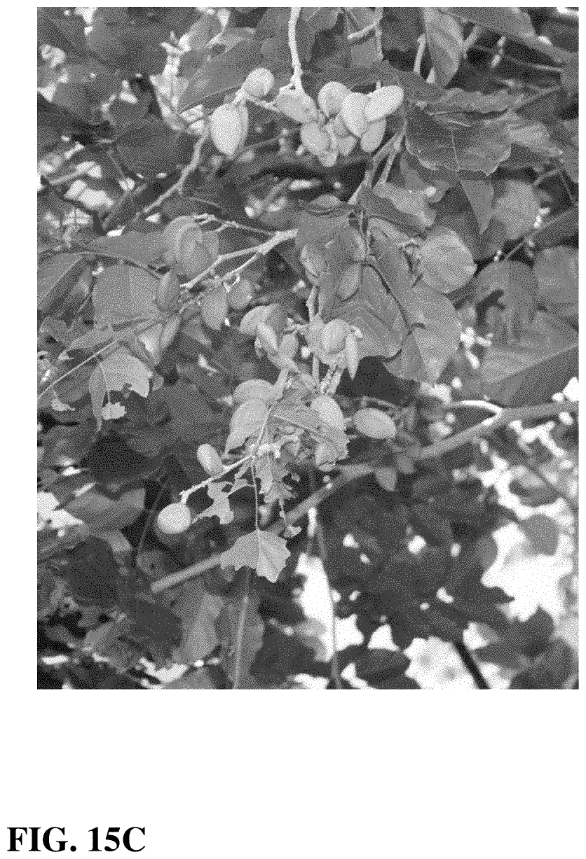

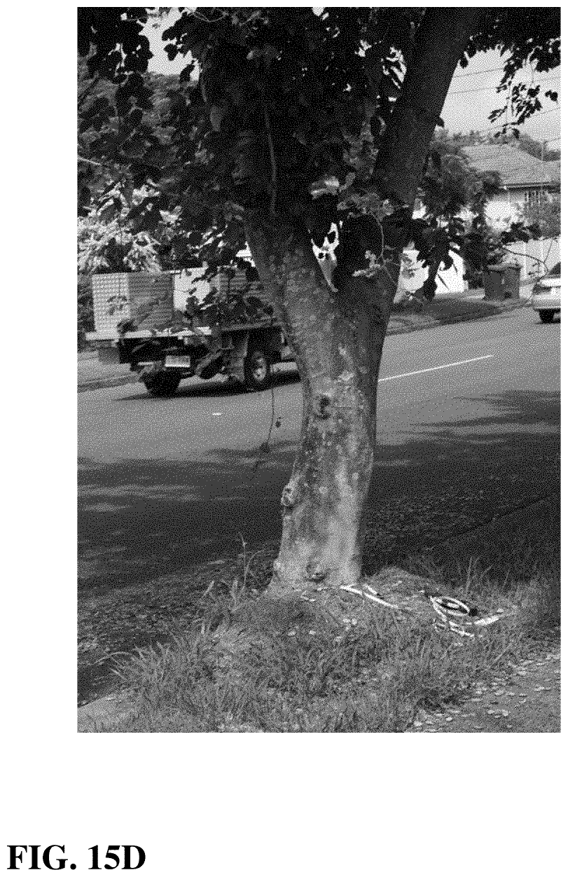

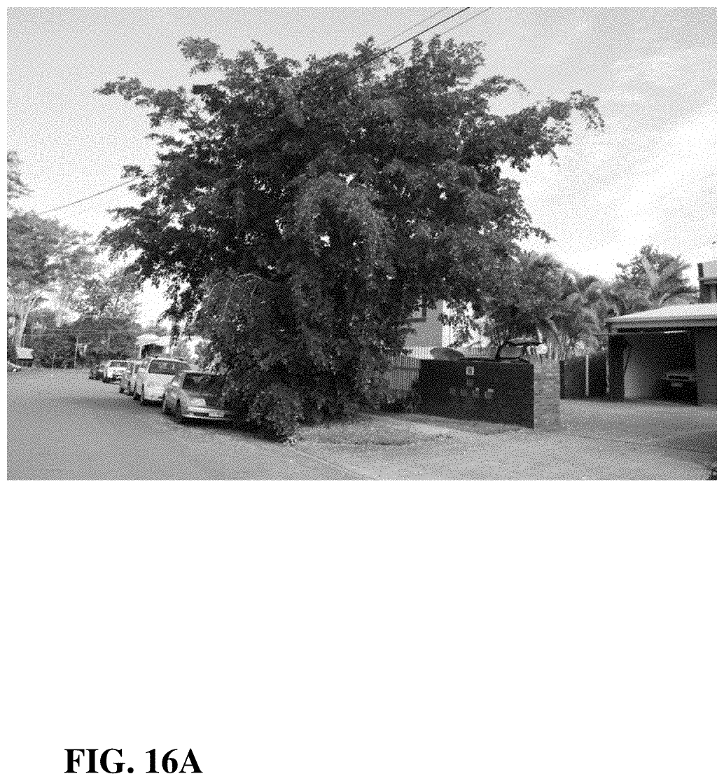

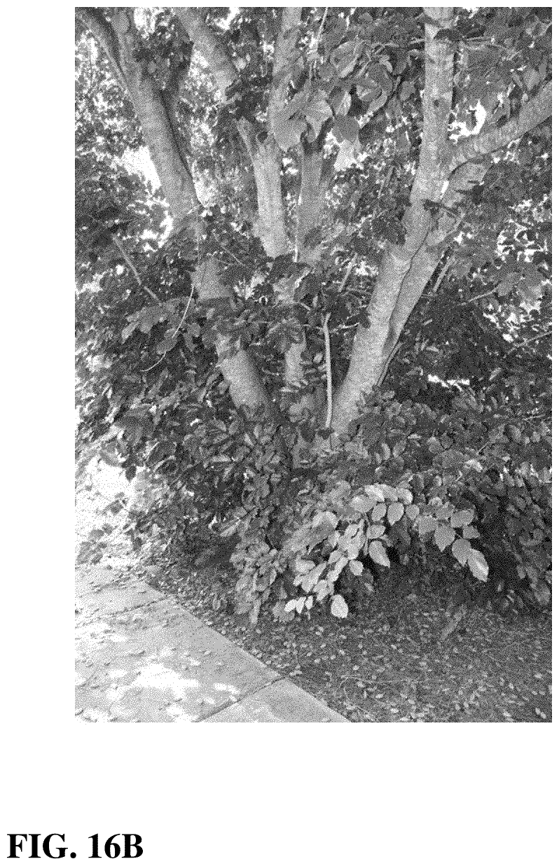

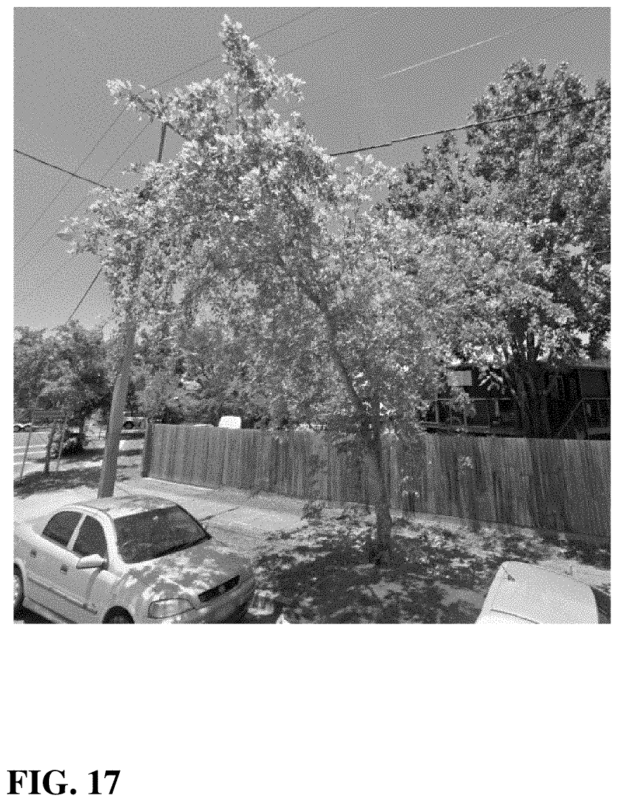

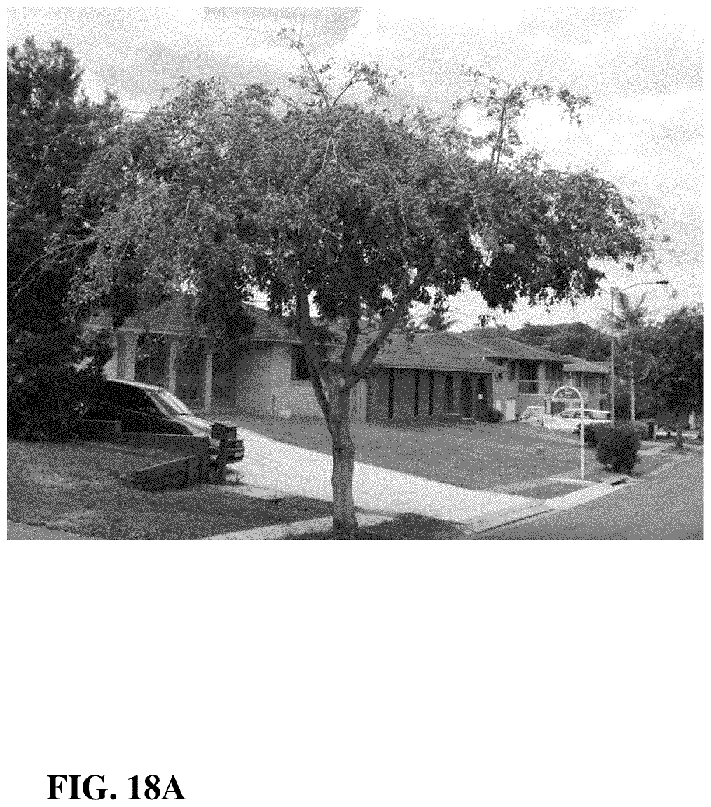

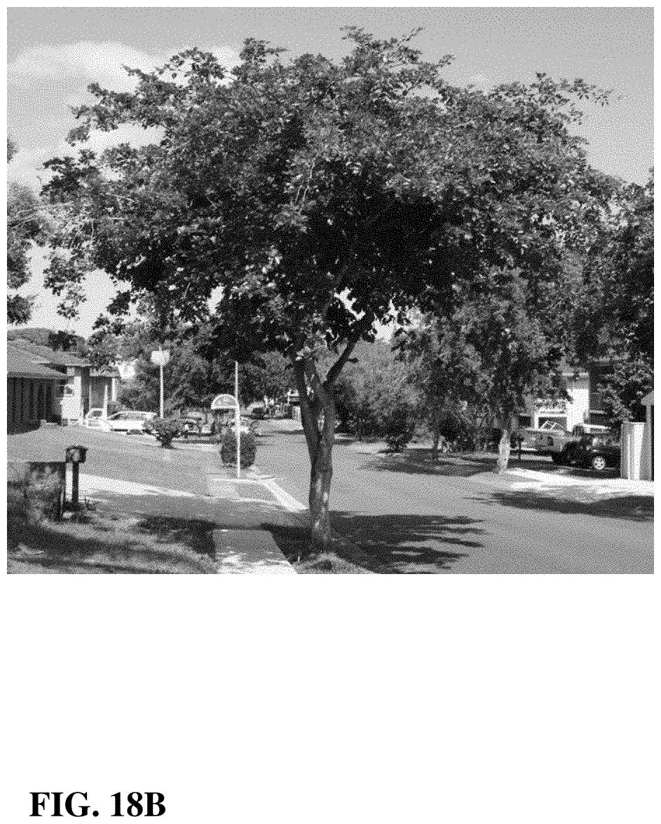

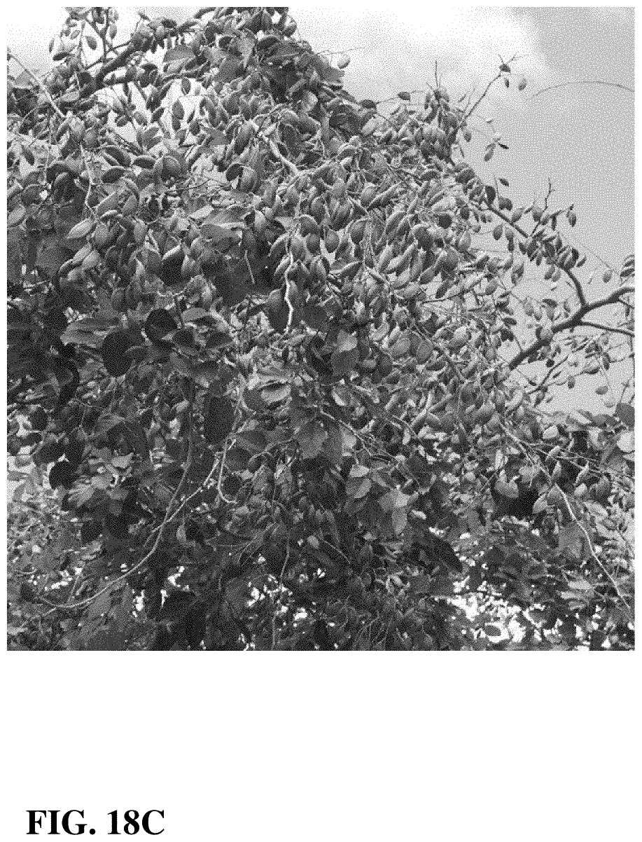

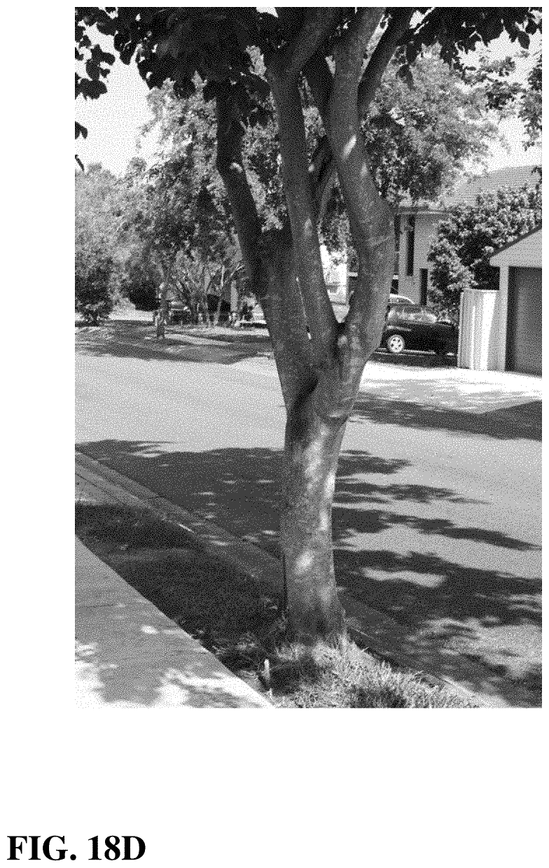

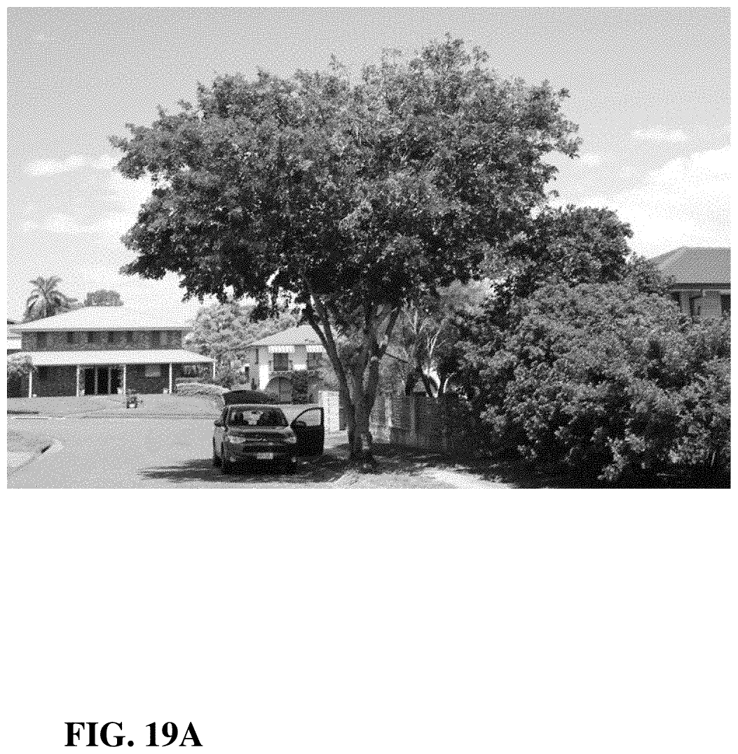

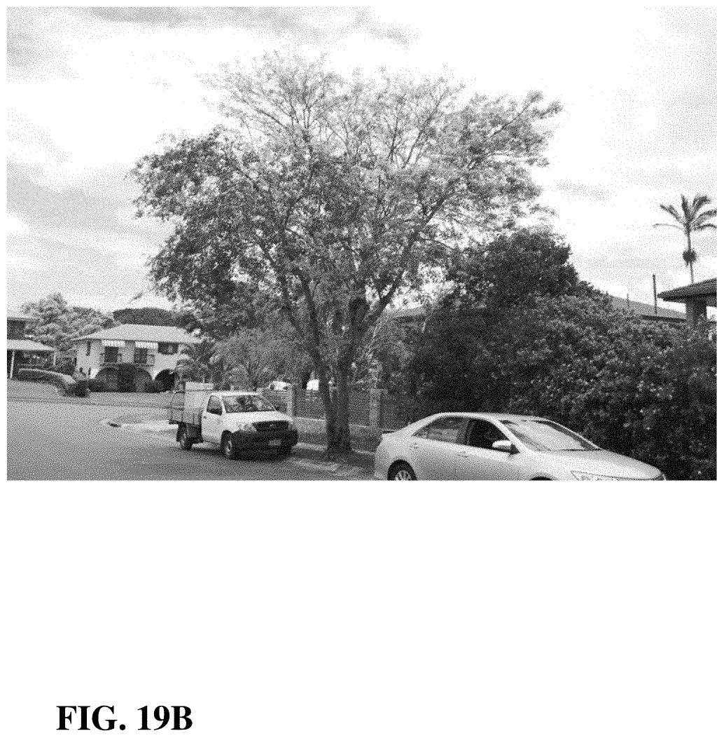

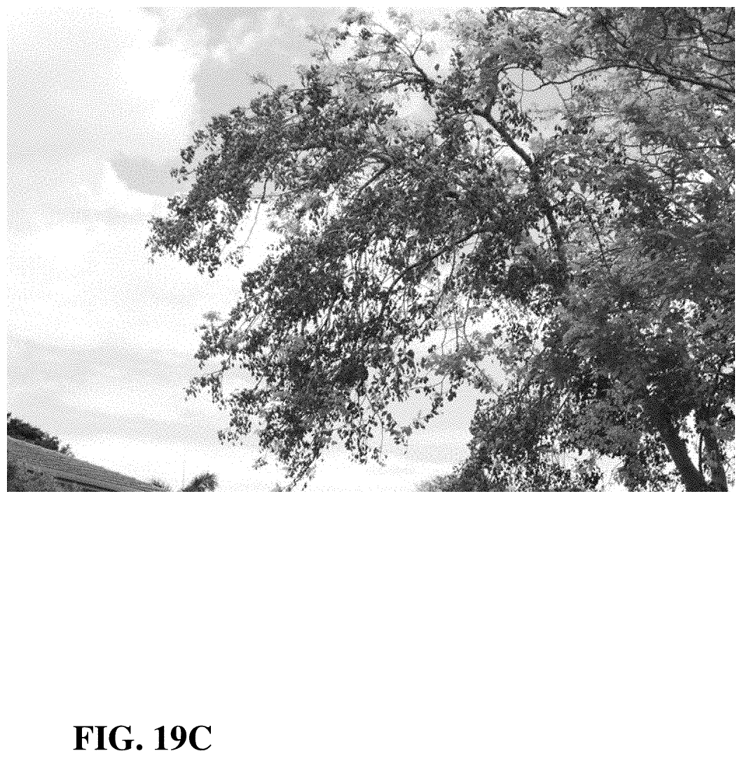

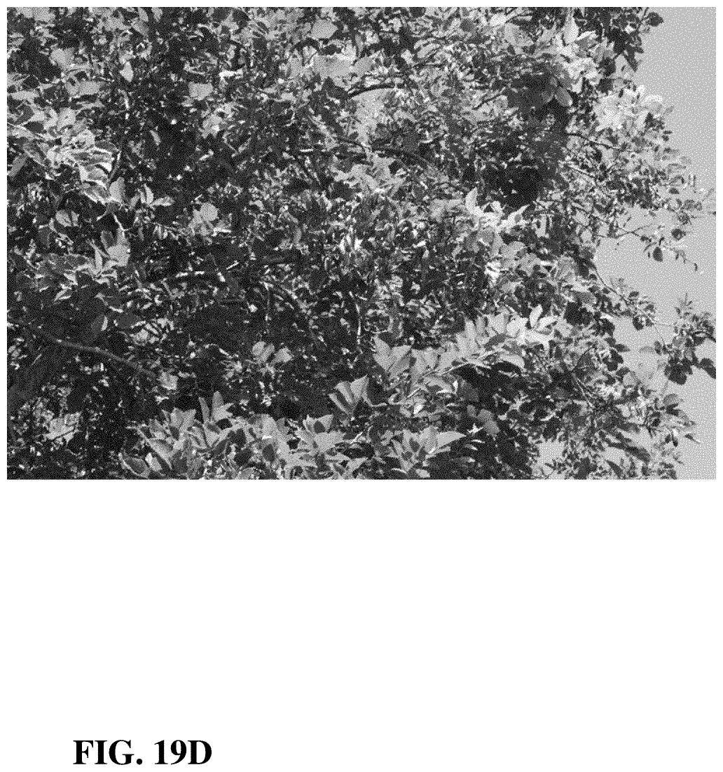

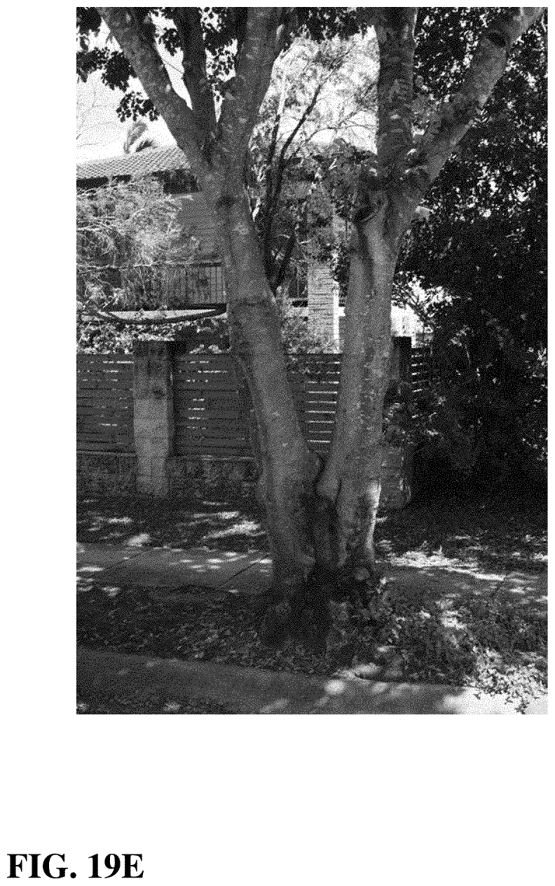

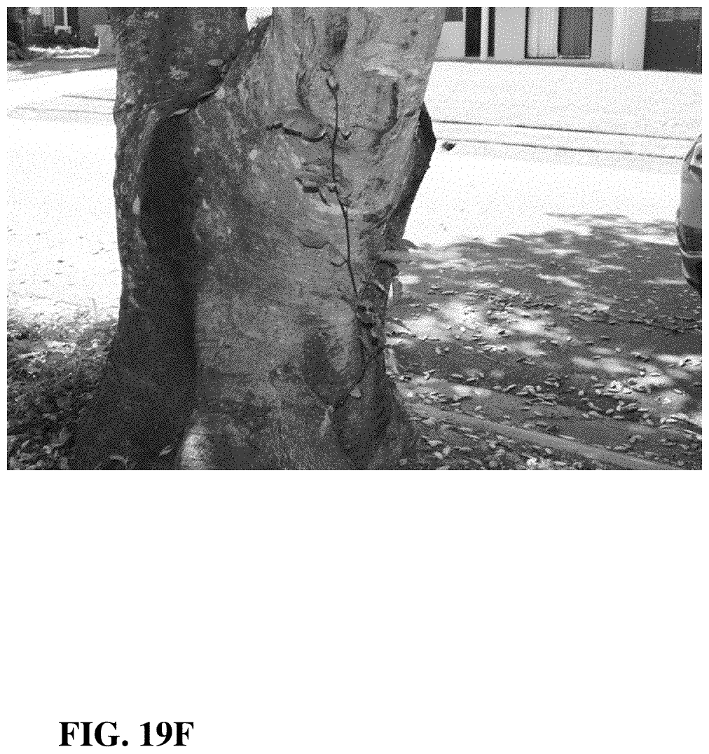

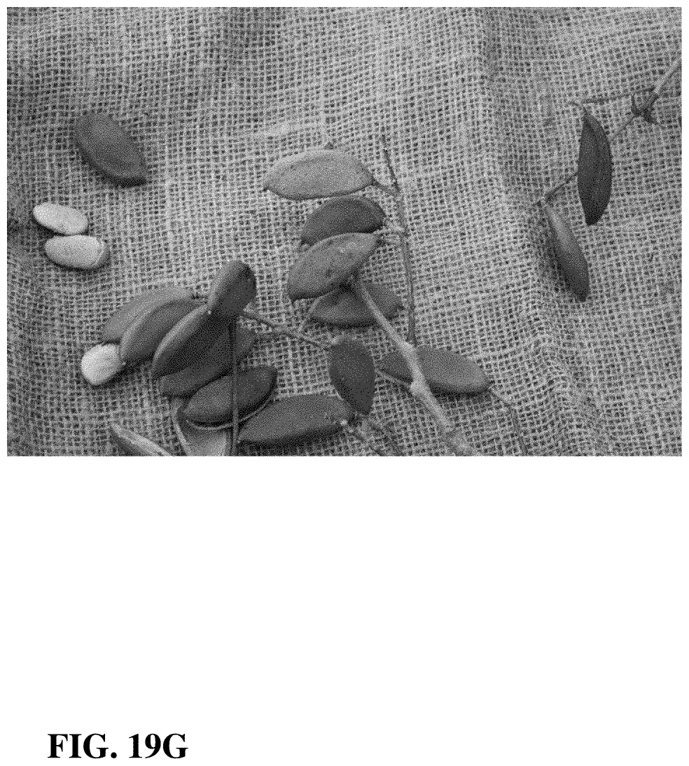

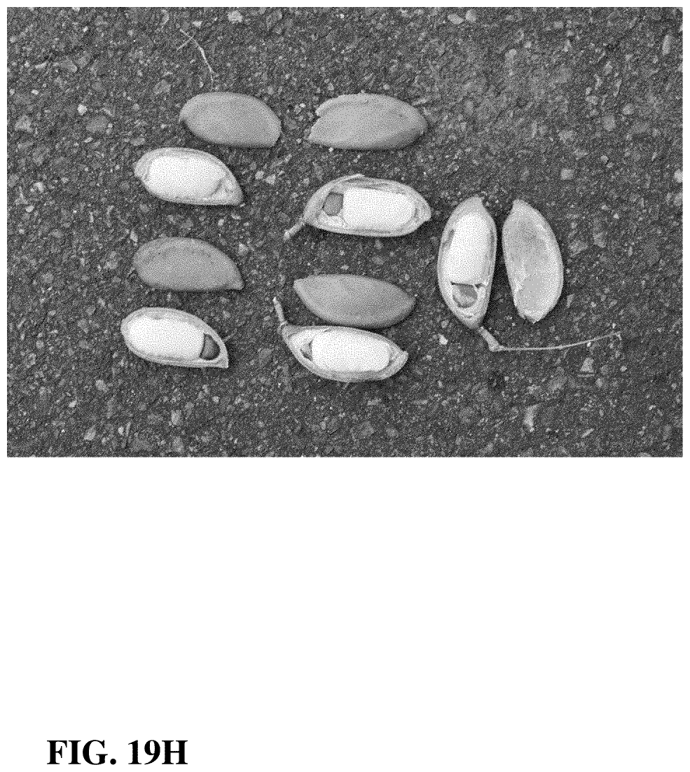

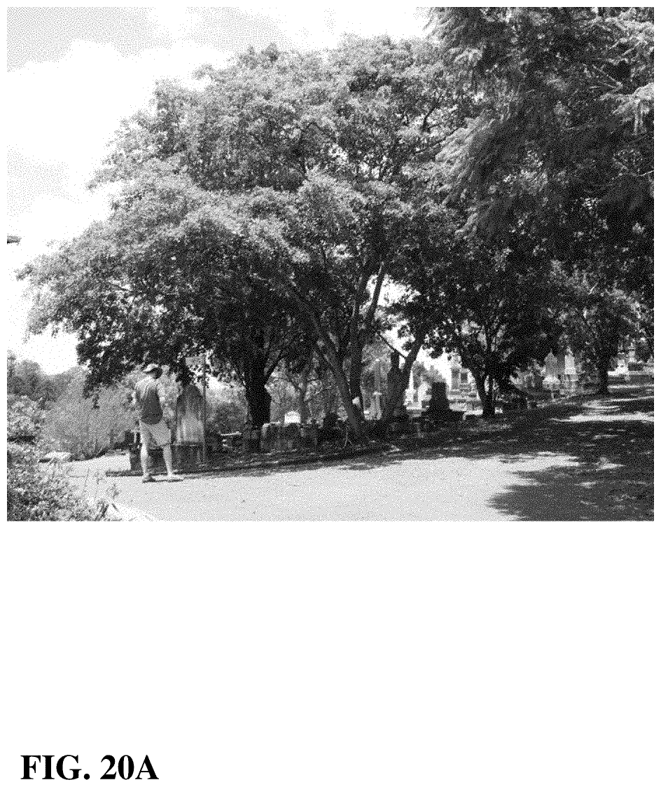

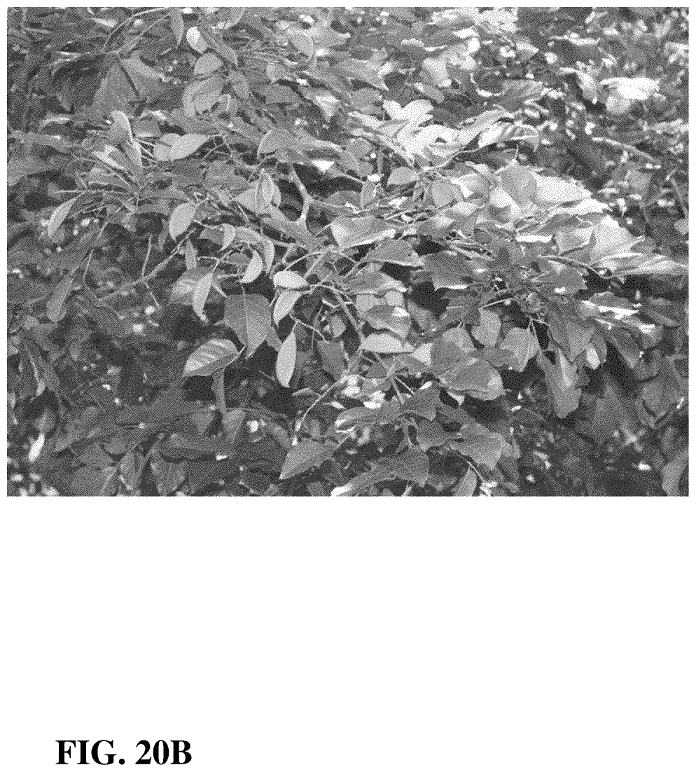

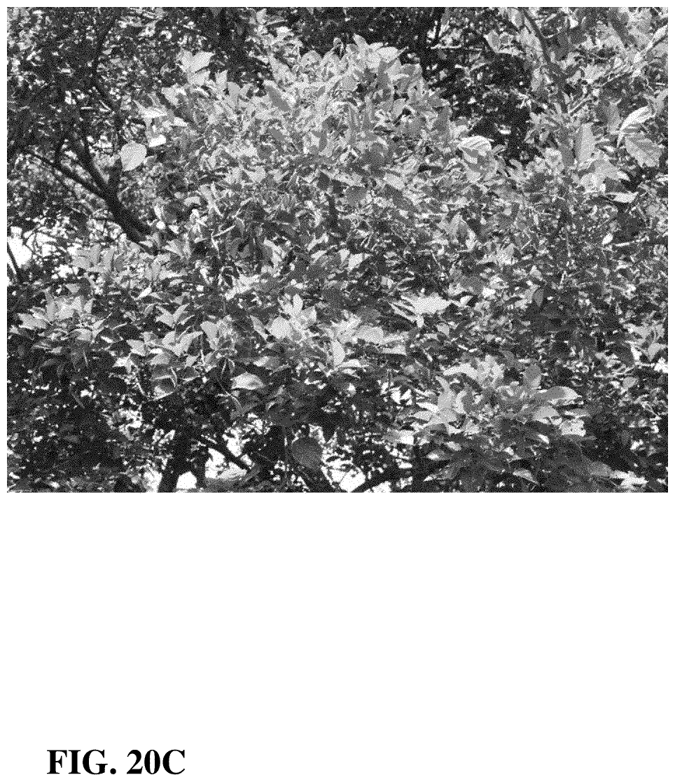

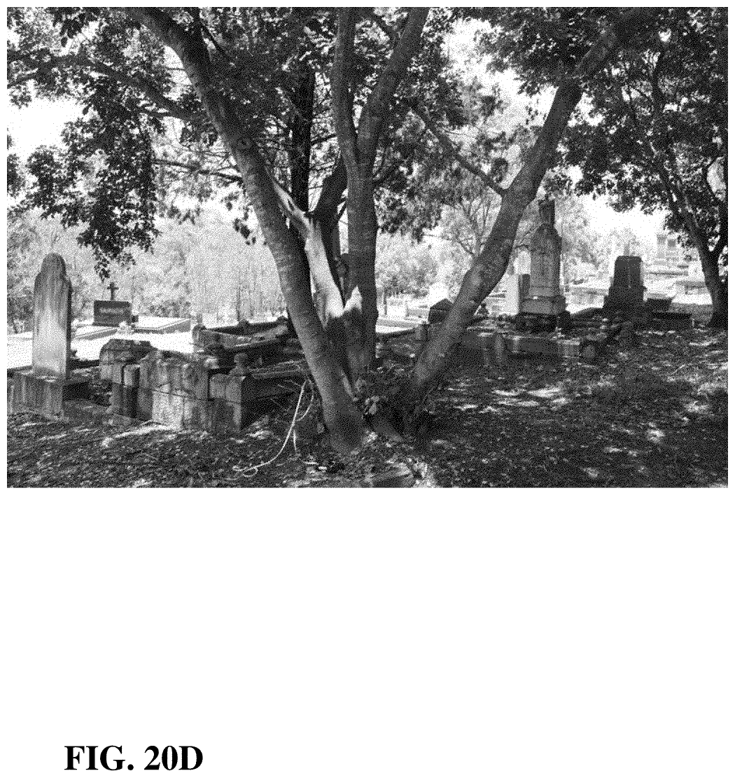

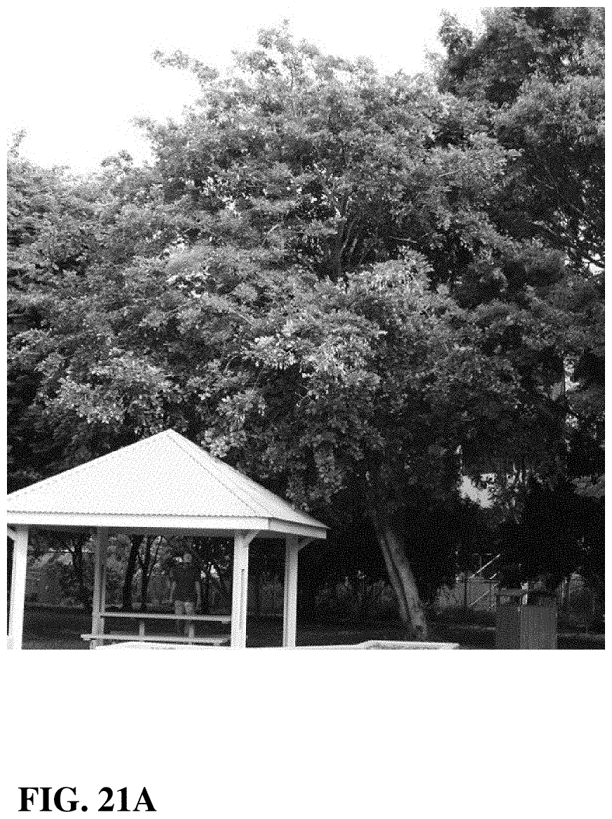

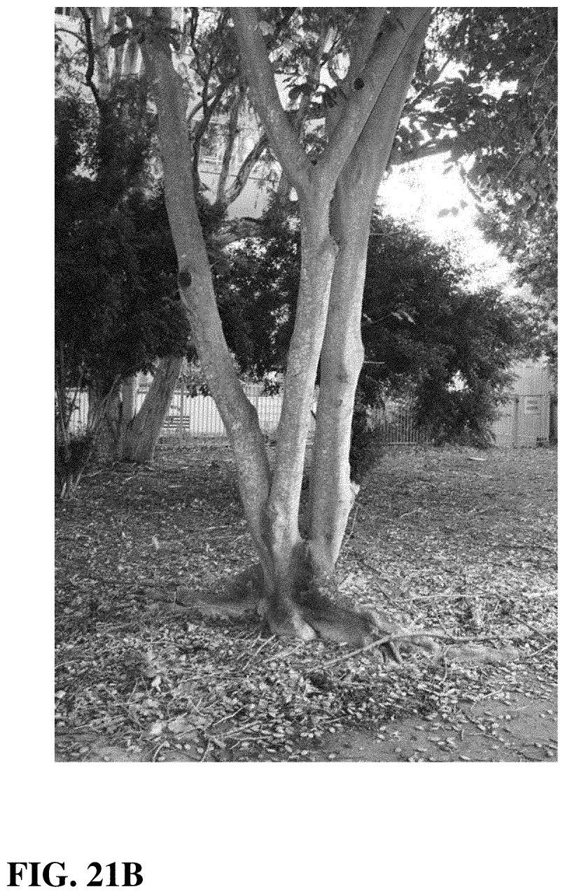

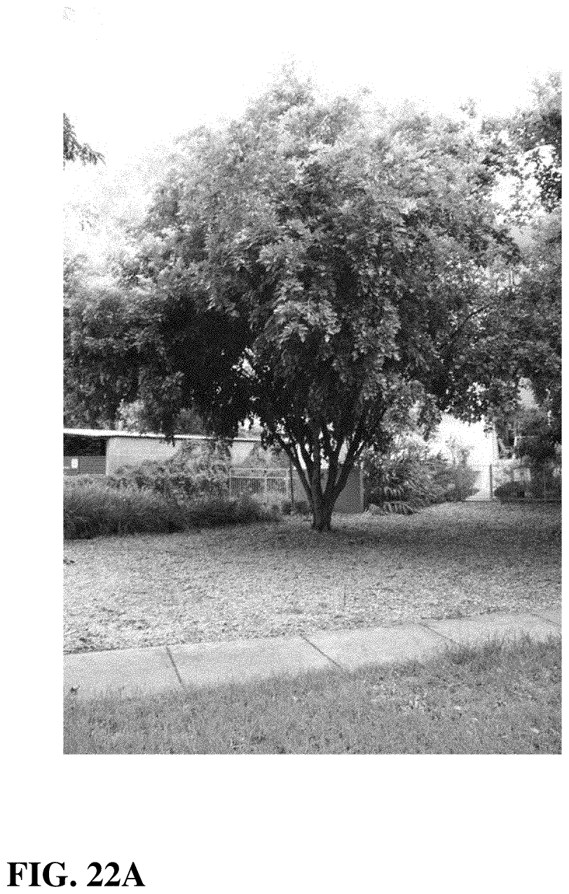

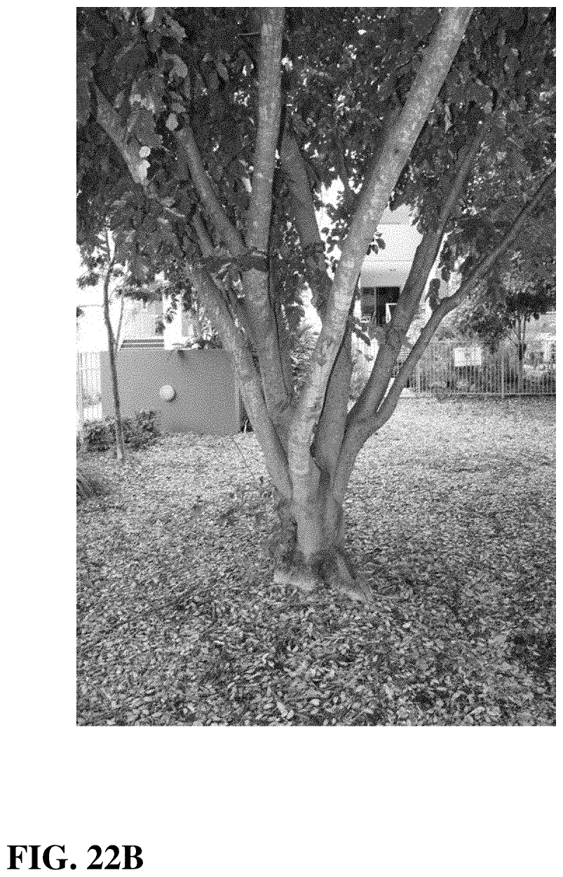

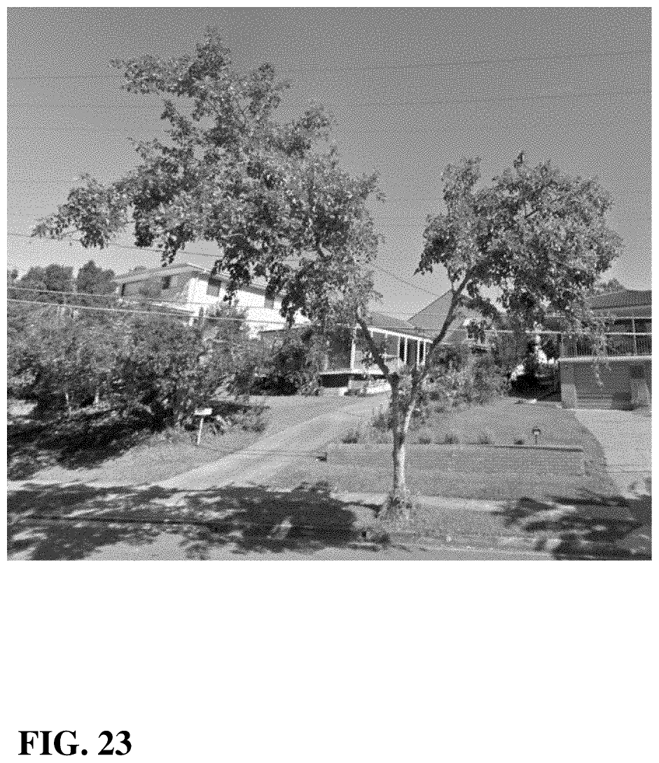

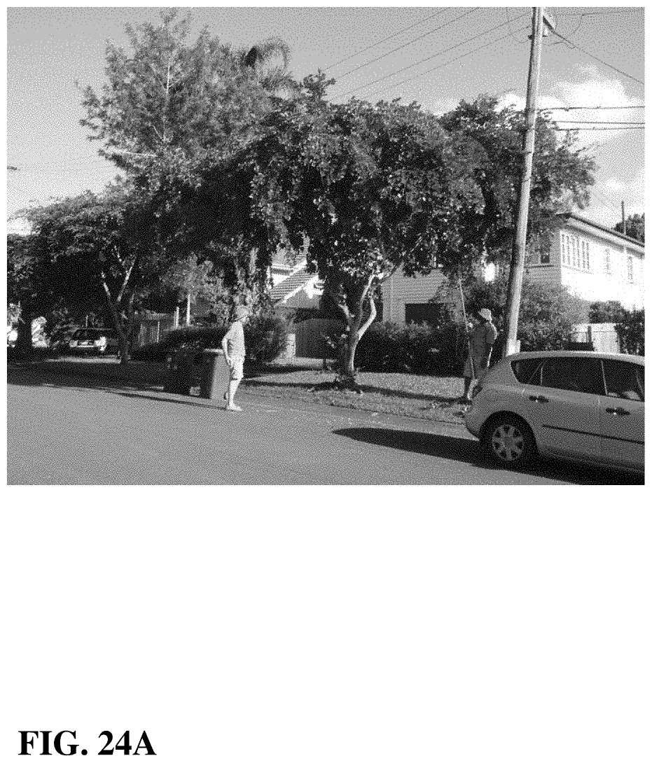

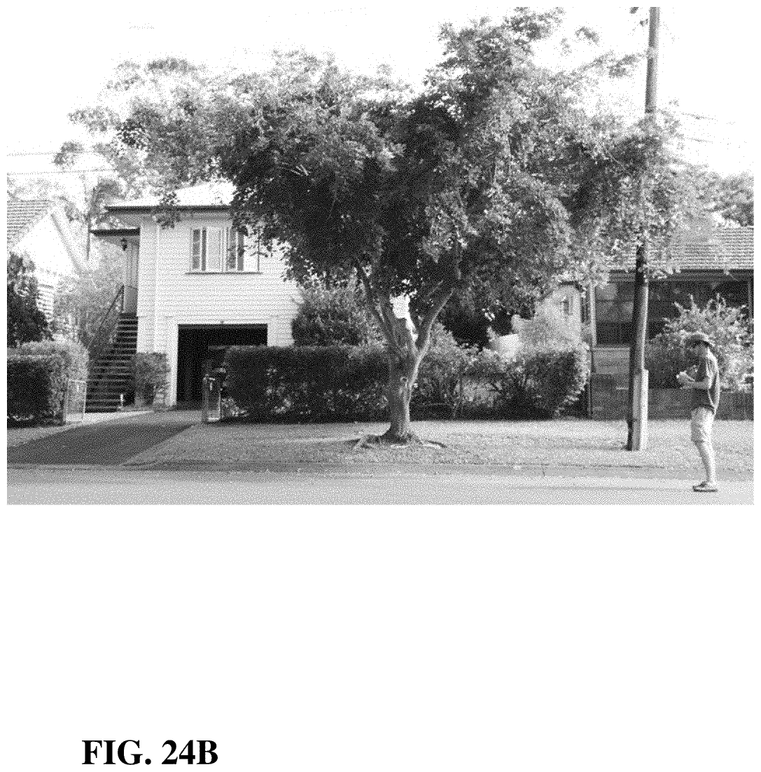

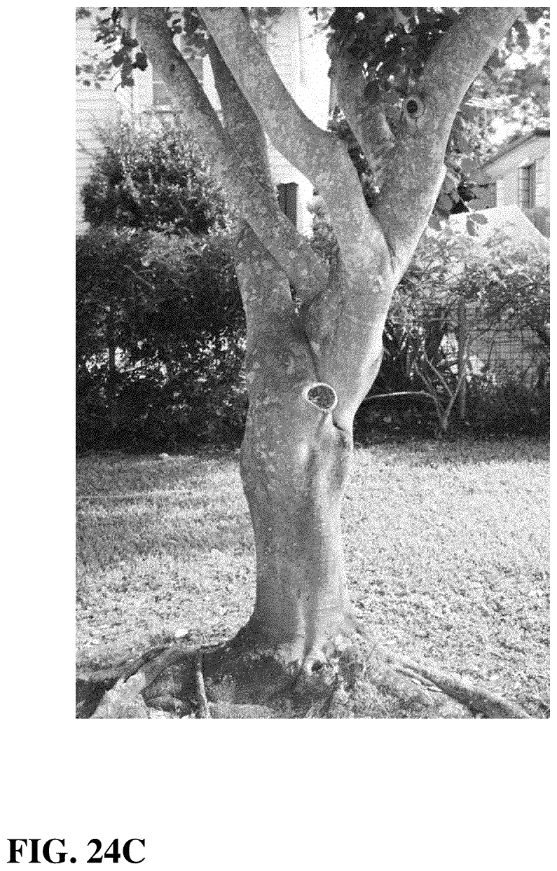

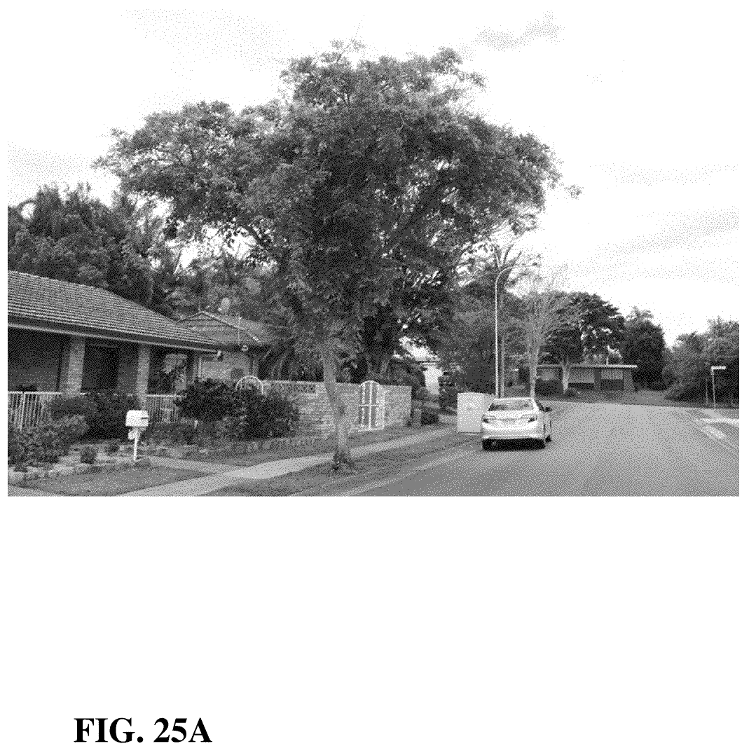

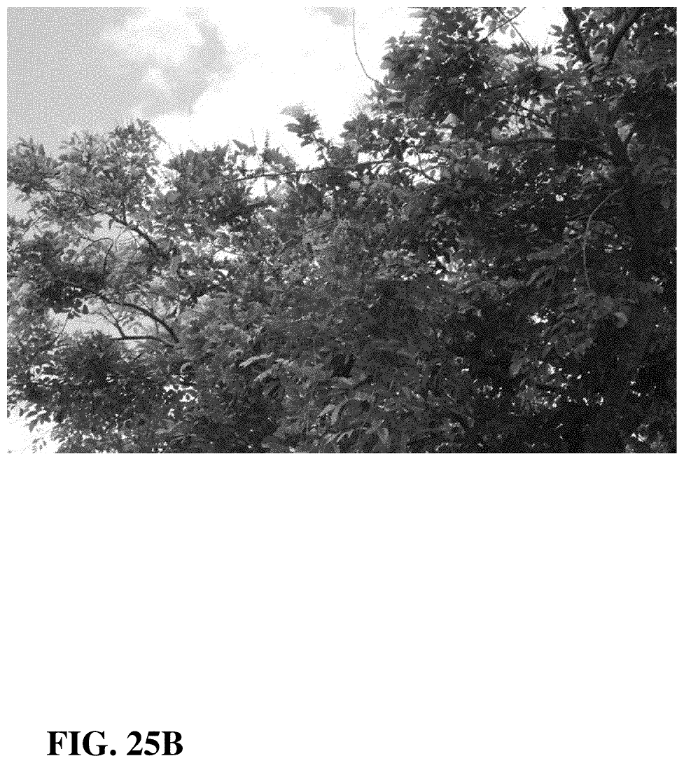

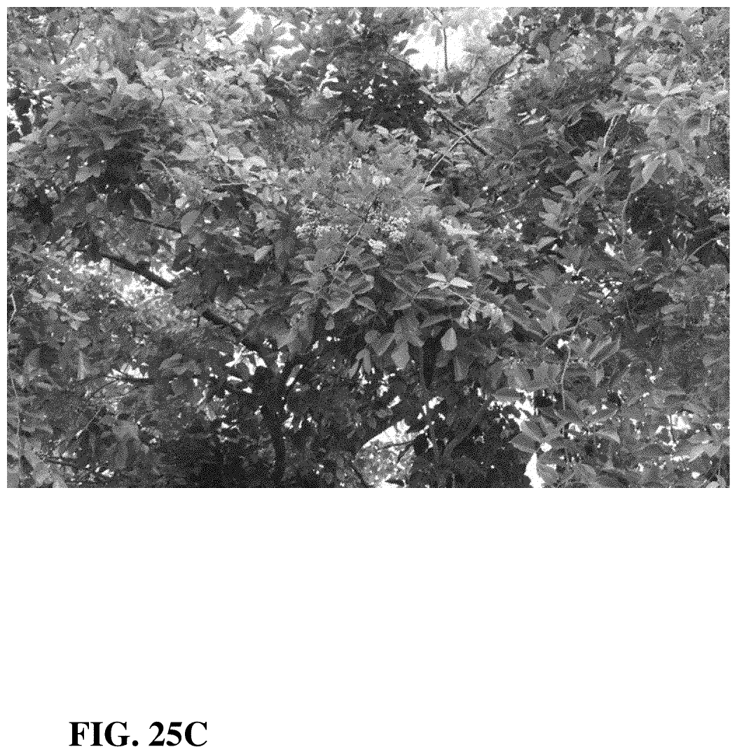

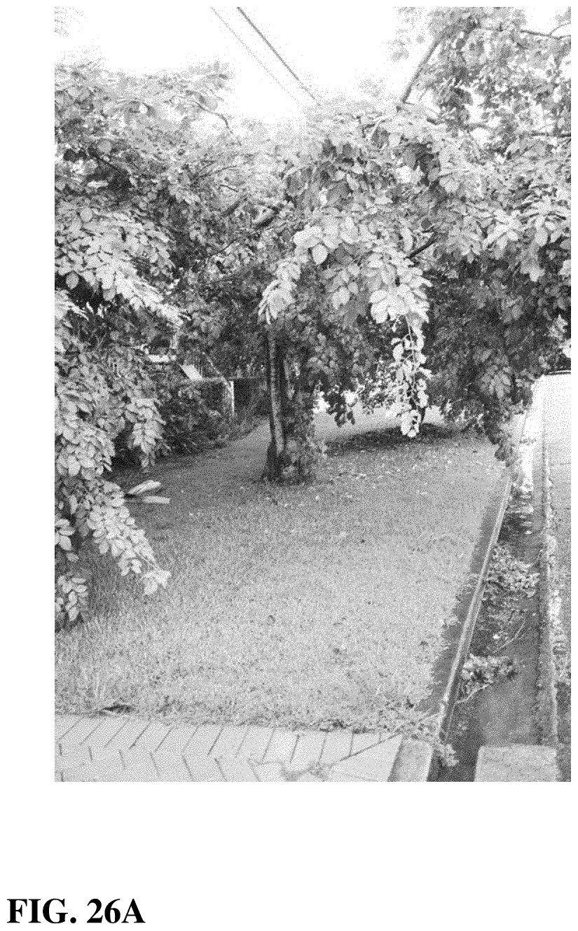

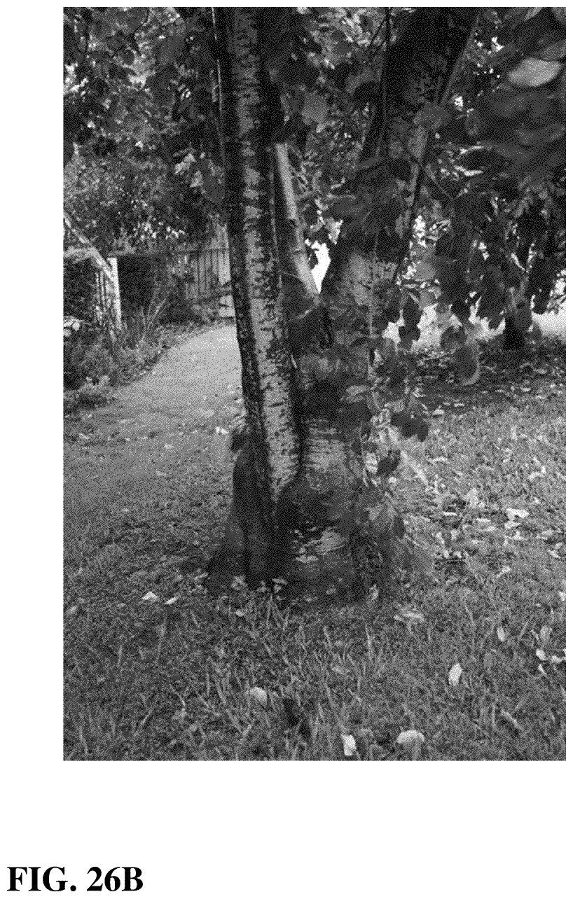

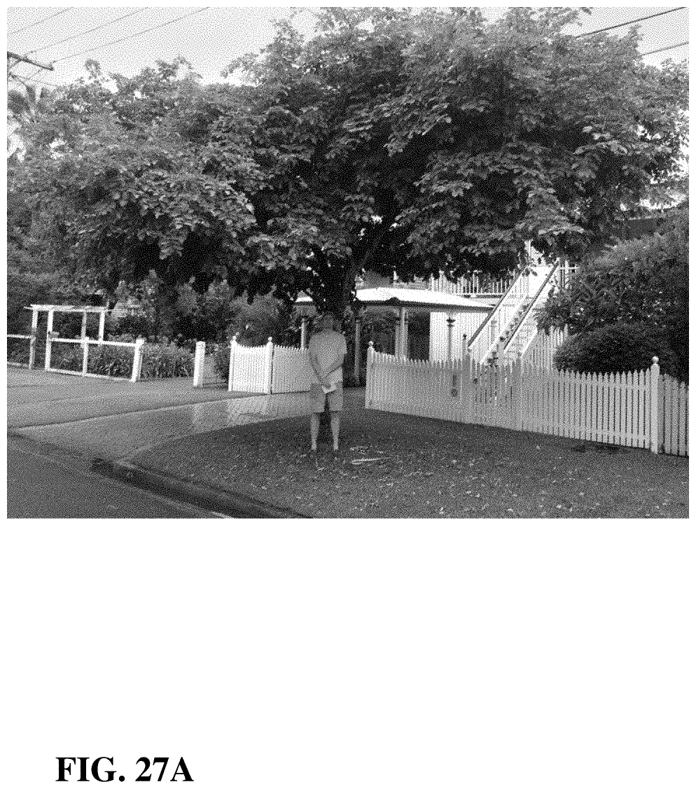

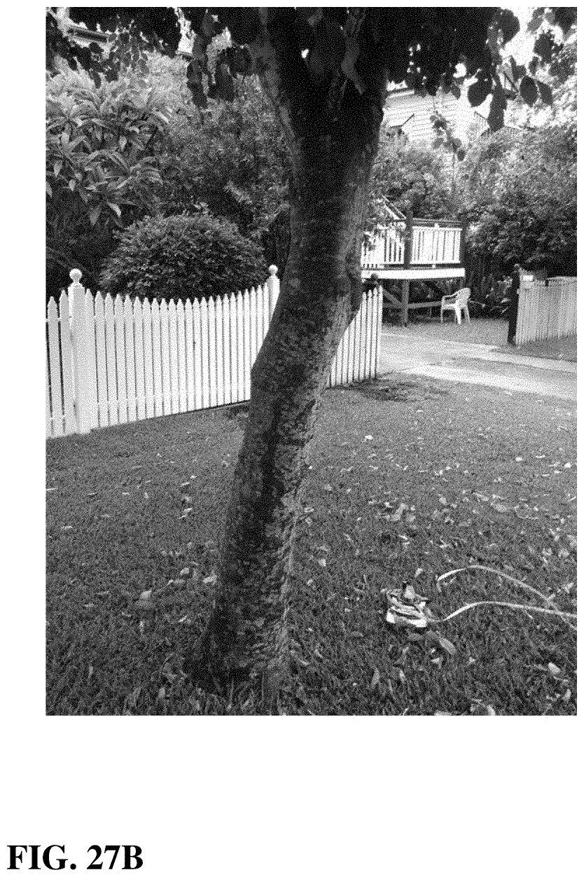

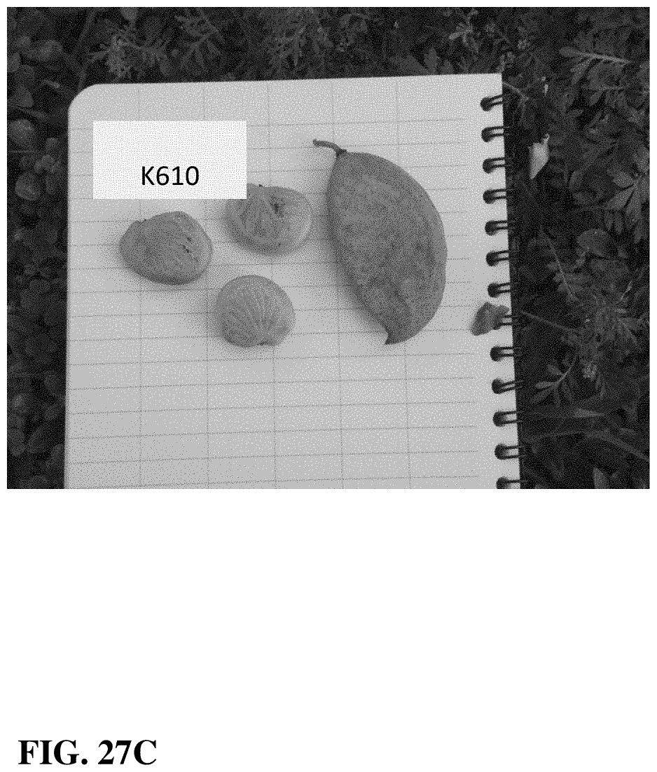

### DETAILED BOTANICAL DESCRIPTION OF THE PLANT

The following detailed description sets forth the phenotypic characteristics and the distinctive features of the pongamia varieties described herein.

### General

The characteristics described below are features of each of ‘K002’, ‘K006’, ‘K008’, ‘K009’, ‘K010’, ‘K011’, ‘K013’, ‘K022’, ‘K025’, ‘K141’, ‘K187’, ‘K202’, ‘K205’, ‘K206’, ‘K207’, ‘K208’, ‘K209’, ‘K211’, ‘K219’, ‘K268’, ‘K303’, ‘K309’, ‘K605’, ‘K607’, ‘K608’, ‘K609’, and ‘K610’.
Classification:
   Family.—Fabaceae.
   Botanical.—Pongamia pinnata (L) Pierre.
   Common name.—Pongam tree, pongam oiltree, or pongamia.
Tree:
   Size.—Modest size and stature.
   Trunk.—Tree crowns and trunks continue to grow as trees age.
   Bark.—Smooth, grey, with some lighter-color mottling. Faint lenticular scars are visible as horizontal striations.
   Form.—Decurrent crown form with dense, compact foliage. Outer branches are arched downward without drooping.
Branches:
   Branch length.—Highly variable year to year due to regular crown management, and thus is not a reliable defining characteristic of the tree.
   Branching habit.—Many branched limbs. Growth and size of lateral branches are considerably less than that of the main branches. Many smaller branches occur higher in the crown.
   Buds and shoots.—Terminal buds are absent. Young shoots emerge from naked lateral buds, with the most distal bud giving rise to a terminal shoot. Buds are hemispherical and 3.3-5.0 mm wide where they attach to the leaf axis. They protrude 1.7-3.5 mm out from the leaf axis and are 1.2-1.5 mm thick. The typical bud color is light green on younger shoots, and greenish-brown on more mature shoots.
   Terminal shoots.—The seasonal elongation of terminal shoots on main branches is considerable.
   Lateral shoots.—Seasonal elongation of lateral shoots is considerably shorter.
   Shoot and twig surfaces.—Woody twigs have leaf scars, showing traces of three vascular bundles. The youngest shoots are distinctly green and flexible, with no lenticels apparent to the naked eye. As shoots mature, their color shifts from green to tan and prominent lenticels become visible as raised light-colored corky circles.
Leaves:
   General.—Pinnately compound leaves bear leaflets. Leaves are arranged alternately along the stem.
   Size and shape.—The outline of a compound leaf is roughly oval.
   Leaflets.—Blades of individual leaflets vary in outline among varieties.
   Leaflet color, surface, and texture.—Leaflets are glabrous on both abaxial and adaxial surfaces. Young leaves exhibit a strikingly glossy cuticle on their adaxial surface. The abaxial surface is less glossy. Leaflets turn a darker shade of green as they age and become increasingly sclerophyllous.
   Stipules.—A pair of small stipules often subtends the youngest leaves on a shoot. They remain for only a short duration before they dehisce.
Flowers and inflorescences:
   General.—Hermaphroditic florets are borne on an indeterminate inflorescence.
   Inflorescence structure.—Several florets are clustered into a fascicle node which are more-or-less evenly distributed along a rachis. The entire inflorescence is a pseudoraceme. Inflorescences can be branched or unbranched.
   Position in crown.—Inflorescences are borne in basal leaf axils of the current year's vegetative flush, near the distal end of the prior year's shoot growth. An average inflorescence typically encompasses over 50 florets. Flower clusters tend to be most common towards the outer surface of the crown.
   Florets.—Emerge acropetally from a fused ovoid calyx with entire margins and glabrous texture. Pedicels are approximately 0.5 mm in diameter. Zygomorphic florets consist of 2 keel, 2 wing, and a standard (or banner) petal. The banner apex is emarginate and the base is rounded. “Wings” exhibit pinkish or whitish coloration on both surfaces. Wing petal apices are obtuse and bases are truncate.
Fruits:
   General.—Seed pods usually bear a single seed, infrequently two seeds.
   Shape and color.—Developing fruits first appear as dark-green flattened pods, expanding in size into lighter green and eventually tan-colored woody pods.
   Crown distribution.—Pods can occur throughout the crown, but are most prevalent towards the outer portions of the crown.
   Fruit clusters.—Pods are connected by the rachis of the inflorescence from which they developed.
   Dimensions.—Pods shapes are distinctive and vary among varieties.
Seeds:
   General.—Typically borne singly in pods. Sometimes an aborted (and much smaller) seed occurs alongside a fully-formed seed.
   Seed coat.—Seeds are covered by a thin seed coat that is beige to dark-brown.
   Seed oil.—Oil content is higher than average, relative to other pongamia trees.
   Nut crop.—Regular bearer.
   Crop frequency.—Most pongamia trees rarely produce abundant nut crops across multiple years, instead bearing fruit biennially or less frequently.
   Ripening.—If undisturbed, pods remain in the crown for several weeks after they ripen.

### Distinctive Features

Table 1 below outlines several characteristics of the pongamia varieties described herein.

TABLE 1
Data on Pongamia Varieties

| | Seed Oil | Tree | | | | |
|---|---|---|---|---|---|---|
| | (%, Dry- | Height | Stem Diameter | | No. Stems | Crown |
| Variety | wt Basis) | (m) | #1 (cm) | #2 (cm) | (@ 1.4 m) | Width (m) |
| K002 | 39.5 | 8 | 18 | 18 | 5 | 10 |
| K006 | — | 8 | 20 | 19 | 2 | 9 |
| K008 | 39.2 | 6 | 22 | — | 1 | 8 |
| K009 | 41.5 | 7 | 23 | 20 | 5 | — |
| K010 | — | 8 | 26 | — | 1 | 11 |
| K011 | — | 5 | 17 | — | 1 | 7 |
| K013 | — | 5 | 22 | — | 1 | 7 |
| K022 | — | 8 | 29 | — | 1 | 11 |
| K025 | 33.4 | 11 | 22 | 19 | 3 | 11 |
| K141 | — | — | — | — | — | — |
| K187 | 39.7 | 9 | 22 | 17 | 2 | 10 |
| K202 | 43.9 | 8 | 22 | — | 1 | 7 |
| K205 | — | 6 | 20 | 18 | 3 | 9 |
| K206 | 35.5 | 9 | 24 | — | 1 | 10 |
| K207 | 38.9 | 9 | — | — | — | 11 |
| K208 | — | 12 | 24 | 21 | 4 | 15 |
| K209 | — | — | — | — | — | — |
| K211 | 40.2 | — | — | — | — | — |
| K219 | 43.5 | 11 | 29 | 27 | 3 | 14 |
| K268 | 35.8 | 11 | 25 | 24 | 4 | 16 |
| K303 | 35.9 | 11 | 23 | 21 | 3 | 8 |
| K309 | — | 10 | 20 | 16 | 7 | 14 |
| K605 | — | — | — | — | — | — |
| K607 | — | 6 | 16 | 14 | 5 | 9 |
| K608 | — | — | — | — | — | — |
| K609 | — | 6 | 22 | 19 | 5 | 12 |
| K610 | — | 6 | 24 | — | 1 | 11 |

Seed oil data collected from Australia
Tree Height: m, estimated using a clinometer
Stem Diameter: cm, of two largest stems (#1 and #2) at breast height (1.4 m above ground)
Number of Stems: at breast height (1.4 m above ground)
Crown Width: m, average of two measurements

We claim:

1. A new and distinct variety of Pongamia pinnata (L) Pierre tree as shown and described herein, wherein the variety is selected from the group consisting of ‘K002’, ‘K006’, ‘K008’, ‘K009’, ‘K010’, ‘K011’, ‘K013’, ‘K022’, ‘K025’, ‘K141’, ‘K187’, ‘K202’, ‘K205’, ‘K208’, ‘K209’, ‘K211’, ‘K219’, ‘K268’, ‘K303’, ‘K309’, ‘K605’, ‘K607’, ‘K608’, ‘K609’, and ‘K610’.
